(19) United States
(12) Patent Application Publication
(10) Pub. No.: US 2002/0061896 A1
(43) Pub. Date: May 23, 2002

(54) Imidazopyrimidine nucleoside analogues with anti-HIV activity

(76) Inventors: Arshad Siddiqul, Dollard des ormeaux (CA); Alex Cimpoia, Montreal (CA); Patrice Preville, Blainville (CA)

Correspondence Address: Gregory P. LaPoite, Bachman & LaPointe, P.C., Suite 1201, 900 Chapel Street, New Haven, CT, 06510, US

(21) Appl. No.: 09/751,488

(22) Filed: Dec. 28, 2000

Related U.S. Application Data
(60) Provisional application No. 60/173,871, filed on Dec. 30, 1999.

(51) Int. Cl. A61K 31/7064; A61K 31/662; A61K 31/695; A61K 31/519; C07F 7/08; C07F 9/6512

(52) U.S. Cl. 514/258; 514/43; 514/63; 514/80; 544/281; 544/229; 544/244

(57) ABSTRACT

The present invention accordingly provides imidazopyrimidine nucleoside compounds of formula (I)

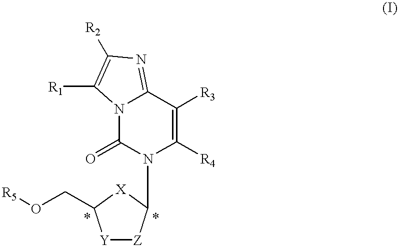

and a method for the treatment or prophylaxis of viral infections in mammals, including humans, comprising the step of administrating a therapeutically effective amount of said compound of formula (I), salts or esters of said compound, pharmaceutical acceptable derivatives or pharmaceutically acceptable salts or esters thereof. Further embodiments include the use of said nucleosides in the preparation of a medicament for the treatment of viral infections in mammals, a commercial packages and pharmaceutical formulations comprising said nucleosides as a therapeutically effective agents against an HIV infection.

The present invention relates to the use of nucleoside analogues in the treatment of viral infections. More specifically it is concerned with the use of imidazopyrimidine nucleoside analogues in the treatment of acquired immunodeficiency syndrome (AIDS).

Retroviral infections are a serious cause of disease, most notably, the acquired immunodeficiency syndrome (AIDS). The human immunodeficiency virus (HIV) has been recognized as the etiologic agent of AIDS. Compounds having an inhibitory effect on HIV multiplication or otherwise effective in the therapy of retroviral infections are being actively sought.

H. Mitsuva et al., “3-Azido-3′-deoxythymidine (BW A509U): An antiviral agent that inhibits the infectivity and cytopathic effect of human T-lymphotropic virus type III/lymphadenopathy-associated virus in vitro”, Proc. Natl. Acad. Sci. USA, 82, pp. 7096-7100 (1985), refers to 3′-azido-3′-deoxythymidine of formula (A), commonly referred to as AZT. This compound is said to be useful in providing some protection for AIDS carriers against the cytopathogenic effect of immunodeficiency virus (HIV).

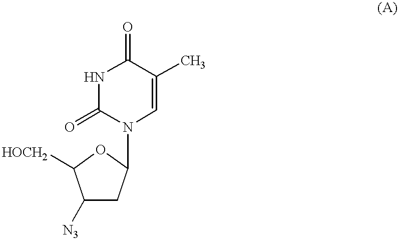

H. Mitsuya and S. Broder, Inhibition of the in vitro infectivity and cytopathic effect of human T-lymphotrophic virus type III/lymphadenopathy-associated virus (HTLV-III/LAV) by 2′,3′-dideoxynucleosides”, Proc. Natl. Acad. Sci. USA, 83, pp.1911-15 (1986), have also referred to a group of 2′,3′-dideoxynucleosides shown in formula (B) which are said to possess protective activity against HIV-induced cytopathogenicity.

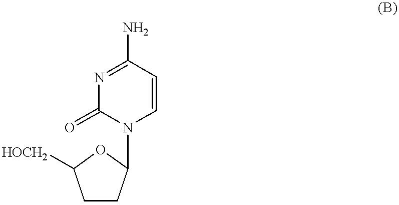

P. Herdewijn et al., “3′-Substituted 2′,3′-dideoxynucleoside analogues as potential anti-HIV(HTLV-III/LAV) agents”, J. Med. Chem., 30, pp. 1270-1278 (1987), describe the anti-HIV activity of a series of 3′-substituted nucleoside analogues. While 3′-fluoro analogues of 3′-deoxythymidine and 2′,3′-dideoxycytidine shown in formulas (C) and (D) are found to possess potent antiretroviral activity, substituents linked to the 3′-carbon via a thio or oxygen bridge did not yield active products.

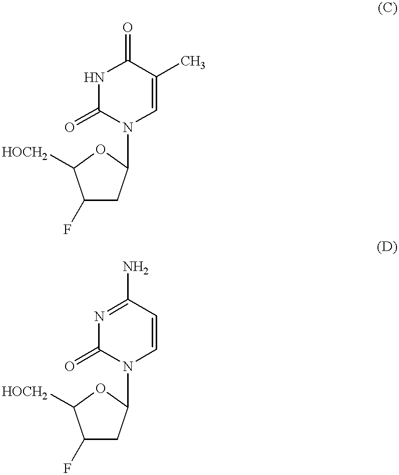

Analysis of molecular conformation studies in P. Van Roey et al., “Correlation between preferred sugar ring conformation and activity of nucleoside analogues against human immunodeficiency virus”, Proc. Natl. Acad. Sci. USA, 86(10), pp. 3929-3933 (1989), indicate that active anti-HIV nucleoside analogues have 3′ carbon conformations on the side opposite to the base.

D. Huryn et al., “Synthesis of iso-ddA, member of a novel class of anti-HIV agents”, Tetrahedron Lett., 30(46), pp. 6259-6262 (1989), refer to the iso-nucleoside analogue of formula (E) as a stable inhibitor of HIV replication.

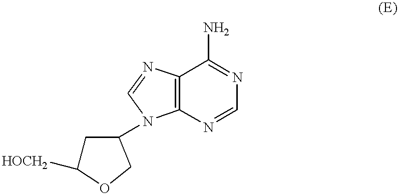

R. Vince and M. Hua, “Synthesis and anti-HIV activity of carbocyclic 2′,3′-didehydro-2′,3′-dideoxy 2,6-disubstituted purine nucleosides, J. Med. Chem., 33(1), pp. 17-21 (1990), describe the analogues shown in formulas (F) and (G) as having anti-HIV activity. The unsaturated analogue (F) shows greater selectivity and potency as an inhibitor of HIV replication than the saturated analog (G).

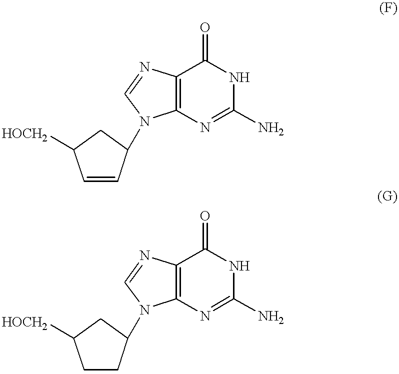

C. Chu et al., “Synthesis and structure-activity relationships of 6-substituted 2′,3′-dideoxypurine nucleosides as potential anti-human immunodeficiency virus agents”, J. Med. Chem., 33(6), pp. 1553-1561 (1990), describe the N₆-methyl derivative shown in formula (H) as having greater potency against HIV than unmethylated 2′,3′-dideoxyadenosine.

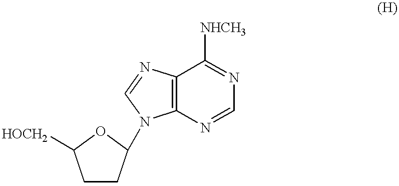

A novel approach by B. Belleau et al. Was described in, “Design and activity of a novel class of nucleoside analogues effective against HIV-1”, Abstracts of papers, Fifth International Conference on AIDS, Montreal, T.C.O. 1, p. 515 (1989), refer to dioxolanes and oxathiolanes of formulas (J) and (K) as having potent anti-HIV activity.

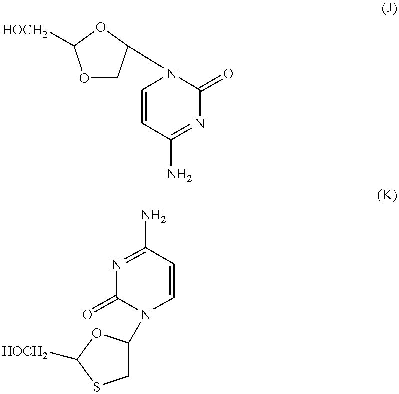

The cis isomer of formula (K) has been found to be active against HIV and HBV, and its unnatural enantiomer ((2R,5S cis) referred to as “the (−) enantiomer” has been found to have surprisingly low toxicity. Now named lamivudine or “3TC™”, this new anti-viral drug is becoming the treatment of choice for combination therapy of AIDS patients and for sole therapy for HBV patients.

Nucleosides analogues of imidazopyrimidines have been reported to be active against Hepatitis B virus and inactive against HIV (Mansour et al (1997a) Bioorg. & Med. Chem. Lett. Vol. 7, No.3: 303-308 and Mansour et al (1997b) Nucleosides & Nucleotides 16 (7-9) 993-1001).

We have now found a new series of nucleoside analogues that consist of an imidazopyrimidine base linked to a sugar ring. These new analogues are active and selective against the human immunodeficiency virus (HIV).

### SUMMARY OF THE INVENTION

The present invention provides nucleoside compounds of formula (I) and a method for the treatment or prophylaxis of viral infections in mammals, including humans, comprising the step of administrating a therapeutically effective amount of said compound of formula (I),

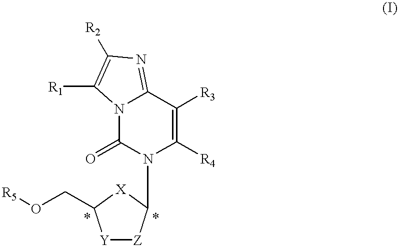

salts or esters of said compound, pharmaceutical acceptable derivatives or pharmaceutically acceptable salts or esters thereof, wherein:

X is oxygen, sulfur or CH₂;

Y and Z are independently selected from sulfur, oxygen, CF₂, C═CH₂ or CH(Rₐ) wherein Rₐ is hydrogen, OH, CN, halogen, N₃, NH₂, SH, C₁₋₆ alkyl, C₁₋₆ alkoxy, C₂₋₆ alkenyl, C₂₋₆ alkynyl, C(O)R_{b}, NHR_{b}, SR_{b} wherein R_{b} is hydrogen, OH, CN, halogen, N₃, NH₂, SH, C₁₋₆ alkyl or C₁₋₆ acyl, C(O)OR_{c} wherein R_{c} is C₁₋₆ alkyl or C₁₋₆ acyl, or Y is CH provided that Z is CH and Y and Z are linked by a double bond;

R₁ is hydrogen, C₁₋₆ alkyl, C₆₋₁₀ aryl or halogen;

R₂ is hydrogen, C₁₋₆ alkyl, C₁₋₆ alcyl, C₆₋₁₀ aryl, C₅₋₁₀ heterocycle, C₂₋₆ acyloxycarbonyl or C₇₋₁₃ aryloxycarbonyl or halogen;

R₃ is hydrogen C₁₋₆ alkyl, C₆₋₁₀ aryl or halogen;

R₄ is hydrogen or halogen;

R₅ is hydrogen; C₁₋₁₀ silylalkyl; C₂₋₁₀ acyl or P(O)(OR₆)O(R′₆) wherein R₆ and R′₆ are independently selected from group hydrogen, C₁₋₆ alkyl, C₂₋₆ alkenyl, C₆₋₁₀ aryl, C₇₋₁₁ arylmethyl, C₂₋₇ acyloxymethyl, C₃₋₈ alkoxycarbonyloxymethyl, C₇₋₁₁ aryloyloxymethyl, C₃₋₈ S-acyl-2-thioethyl; phosphonophosphate or phosphonodiphosphate.

The present invention further provides a method of treatment of and HIV infection in mammals, including humans, comprising the step of administrating a therapeutically effective amount of said compound of formula (I).

A further embodiment of the present invention provides the use of a compound of formula (I) in the manufacture of a medicament for the treatment of human immunodeficiency virus.

The present invention also includes a commercial package containing a pharmaceutical agent comprising one or more compounds of formula (I) or the pharmaceutically acceptable salts or esters thereof.

### DETAILED DESCRIPTION OF THE INVENTION

The present invention accordingly provides imidazopyrimidine nucleoside compounds of formula (I)

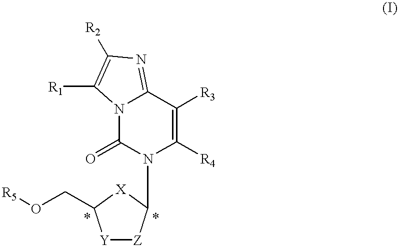

and a method for the treatment or prophylaxis of viral infections in mammals, including humans, comprising the step of administrating a therapeutically effective amount of said compound of formula (I), salts or esters of said compound, pharmaceutical acceptable derivatives or pharmaceutically acceptable salts or esters thereof, wherein:

X is oxygen, sulfur or CH₂;

Y and Z are independently selected from sulfur, oxygen, CF₂, C═CH₂ or CH(Rₐ) wherein Rₐ is hydrogen, OH, CN, halogen, N₃, NH₂, SH, C₁₋₆ alkyl, C₁₋₆ alkoxy, C₂₋₆ alkenyl, C₂₋₆ alkynyl, C(O)R_{b}, NHR_{b}, SR_{b} wherein R_{b} is hydrogen, OH, CN, halogen, N₃, NH₂, SH, C₁₋₆ alkyl or C₁₋₆ acyl, C(O)OR_{c} wherein R_{c} is C₁₋₆ alkyl or C₁₋₆ acyl, or Y is CH provided that Z is CH and Y and Z are linked by a double bond;

R₁ is hydrogen, C₁₋₆ alkyl, C₆₋₁₀ aryl or halogen;

R₂ is hydrogen, C₁₋₆ alkyl, C₁₋₆ alcyl, C₆₋₁₀ aryl, C₅₋₁₀ heterocycle, C₂₋₆ acyloxycarbonyl or C₇₋₁₃ aryloxycarbonyl or halogen;

R₃ is hydrogen C₁₋₆ alkyl, C₆₋₁₀ aryl or halogen;

R₄ is hydrogen or halogen;

R₅ is hydrogen; C₁₋₁₀ silylalkyl; C₂₋₁₀ acyl or P(O)(OR₆)O(R′₆) wherein R₆ and R′₆ are independently selected from group H, C₁₋₆ alkyl, C₂₋₆ alkenyl, C₆₋₁₀ aryl, C₇₋₁₁ arylmethyl, C₂₋₇ acyloxymethyl, C₃₋₈ alkoxycarbonyloxymethyl, C₇₋₁₁ aryloyloxymethyl, C₃₋₈ S-acyl-2-thioethyl; phosphonophosphate or phosphonodiphosphate.

The term “alkyl”, as used herein, unless otherwise specified, refers to a saturated straight, branched, or cyclic, primary, secondary, or tertiary hydrocarbon of C₁₋₃₀, particularly C₁₋₆, unsubstituted or optionally mono- or di-substituted by hydroxy, N₃, CN, SH, amino, halogen (F, Cl, Br, I), C₆₋₁₂ aryl, C₁₋₆ alkyl, C₂₋₁₂ alkoxyalkyl or nitro. It specifically includes methyl, ethyl, cyclopropyl, propyl, isopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl.

The term “acyl,” as used hereinafter, refers to a radical derived from an aliphatic carboxylic acid, by removal of the —OH group of 1 to 30 carbon atoms, particularly 1 to 6 carbon atoms. Like the acid to which it is related, an aliphatic acyl radical may be substituted (by a hydroxy, amino, N₃, CN, halogen (F, Cl, Br, I), C₆₋₁₂ aryl, C₁₋₆ alkyl, C₂₋₁₂ alkoxyalkyl or nitro) or unsubstituted, and whatever the structure of the rest of the molecule may be, the properties of the functional group remain essentially the same (e.g. acetyl, propionyl, isobutanoyl, pivaloyl, hexanoyl, butyryl, pentanoyl, 3-methylbutyryl, hydrogen succinate, mesylate, valeryl, caproic, caprylic, capric, lauric, myristic, palmitic, stearic, oleic, 3-chlorobenzoate, trifluoroacetyl, chloroacetyl, and cyclohexanoyl).

The terms “alkenyl” and “alkynyl” represent substituted (by a N₃, CN, halogen, hydroxyl or C₆₋₂₀ aryl) or unsubstituted straight, branched or cyclic hydrocarbon chains having 2 to 30 carbon atoms and preferably from 2 to 6 carbon atoms and containing at least one unsaturated group (e.g. allyl).

The term “alkoxy” represents a substituted or unsubstituted alkyl group containing from 1 to 30 carbon atoms and preferably from 1 to 6 carbon atoms, wherein the alkyl group is covalently bonded to an oxygen atom (e.g., methoxy and ethoxy).

The term “aryl” represents a aromatic moiety which may be substituted by hydroxy, N₃, CN, halogen (F, Cl, Br, I), amino and containing at least one benzenoid-type ring, the group may contain from 6 to 14 carbon atoms (e.g., phenyl and naphthyl), particularly 6 to 10 carbon atoms.

The term “aryloxy” represents a substituted (by a halogen, trifluoromethyl or C₁₋₅ alkoxy) or unsubstituted aryl moiety, having 6 to 14 carbon atoms, covalently bonded through an oxygen atom (e.g., benzyloxy, phenoxy).

The term “arylalkyl” represents a substituent comprising an aryl moiety attached via an alkyl chain (e.g. benzyl, phenylethyl) wherein the sum total of carbon atoms for the aryl moiety and the alkyl chain is 7 to 21. The aryl or chain portion of the group is optionally mono- or di-substituted with OH, SH, amino, halogen or C₁₋₆ alkyl.

The term “thiol” represents C₁₋₆ alkyl, C₆₋₁₅ aryl, C₇₋₂₁ aralkyl, C₂₋₆ alkenyl or C₂₋₆ alkynyl groups covalently bonded to an adjacent sulfur atom bearing a hydrogen.

The terms “alkylthio” (e.g. thiomethy, thioethyl) and “arylthio” (e.g. thiophenyl, thiobenzyl), refers to C₁₋₆ alkyl or C₆₋₁₀ aryl groups, unsubstituted or optionally mono- or di-substituted by hydroxy, halogen (F, Cl, Br, I), C₆₋₁₂ aryl, C₁₋₆ alkyl, C₂₋₁₂ alkoxyalkyl or nitro, covalently bonded to an adjacent sulfur atom.

The terms “acyloxy” and “alkoxycarbonyl” refer to 2 to 30 carbon atoms chains, particularly 2 to 10 carbon atoms, that are either saturated or unsaturated and may also be straight or branched covalently bonded to an oxygen atom (e.g.: acetyloxy). The chains may be unsubstituted or optionally mono- or di-substituted by hydroxy, N₃, CN, SH, amino, halogen (F, Cl, Br, I), C₆₋₁₂ aryl, C₁₋₆ alkyl, C₂₋₁₂ alkoxyalkyl or nitro.

The term “alkoxyalkyl” represents a C₁₋₆ alkoxy group attached to an adjacent C₁₋₆ alkyl group (e.g., methoxymethyl, ethoxymethyl). They may be unsubstituted or optionally mono- or di-substituted by hydroxy, N₃, CN, SH, amino, halogen (F, Cl, Br, I), C₆₋₁₂ aryl, C₁₋₆ alkyl, C₂₋₁₂ alkoxyalkyl or nitro.

The term “heterocycle” represents a saturated or unsaturated mono- or polycyclic (i.e. bicyclic) ring incorporating 1 or more (i.e. 1-4) heteroatoms selected from N, O and S. It is understood that a heterocycle is optionally mono- or di-substituted with OH, SH, amino, halogen, CF₃, oxo or C₁₋₆ alkyl. Examples of suitable monocyclic heterocycles include but are not limited to pyridine, piperidine, pyrazine, piperazine, pyrimidine, imidazole, thiazole, oxazole, furan, pyran and thiophene. Examples of suitable bicyclic heterocycles include but are not limited to indole, benzimidazole, quinoline, isoquinoline, purine, and carbazole.

The term “aralkyl” represents a substituent comprising a C₆₋₁₀ aryl moiety attached via a C₁₋₆ alkyl chain (e.g. benzyl, phenethyl). The aryl or chain portion of the group is unsubstituted or optionally mono- or di-substituted by hydroxy, N₃, CN, SH, amino, halogen (F, Cl, Br, I), C₆₋₁₂ aryl, C₁₋₆ alkyl, C₂₋₁₂ alkoxyalkyl or nitro.

The term amino represents C₁₋₆ alkyl, C₆₋₁₀ aryl, C₂₋₆ alkenyl, C₂₋₆ alkynyl, or C₇₋₁₂ aralkyl groups, unsubstituted or optionally mono- or di-substituted by hydroxy, N₃, CN, SH, amino, halogen (F, Cl, Br, I), C₆₋₁₂ aryl, C₁₋₆ alkyl, C₂₋₁₂ alkoxyalkyl or nitro, wherein the carbon atoms are covalently bonded to an adjacent element through a nitrogen atom (e.g., pyrrolidine). They include primary, secondary and tertiary amines and quaternary ammonium salts. Amino includes NR₈R₉ wherein R₈ and R₉ are independently selected from C₁₋₆ alkyl, C₆₋₁₆ aryl, C₂₋₆ alkenyl, C₂₋₆ alkynyl or C₇₋₁₈ aralalkyl optionally substituted with COOH, C(O)NH₂, OH, SH, NH₂, NO₂, CN or halogen and optionally interrupted by one or more carbonyl or sulfonyl; R₈R₉ can also be connected to the nitrogen atom to form a saturated or unsaturated C₃₋₈ heterocyclic ring optionally substituted with COOH, C(O)NH₂, OH, SH, NH₂, NO₂, halogen, C₁₋₆ alkyl, C₂₋₆ alkenyl or C₂₋₆ alkynyl optionally substituted with COOH, C(O)NH₂, OH, SH, NH₂, NO₂, CN or halogen.

In an alternative embodiment of the present invention the method of treatment of a viral infection in mammals, including humans, which includes the step of the administration of a therapeutically effective amount of compounds of formula (I) wherein R₁ and R₄ are hydrogen; R₅ is hydrogen or C₁₋₆ acyl; X is oxygen or sulfur; and is CH₂. Particularly when X is oxygen.

An alternative embodiment consists of a method of treatment of a viral infection in mammals, including humans, which includes the step of the administration of a therapeutically effective amount of compounds of formula (I) wherein R₁ and R₄ are hydrogen; R₅ is hydrogen or C₂₋₁₀ acyl; X is oxygen; Z is CH₂; and R₂ is hydrogen, C₆₋₁₀ aryl, C₅₋₁₀ heterocycle, C₁₋₆ alkyl, C₂₋₆ acyloxycarbonyl or halogen. The invention further includes compounds of formula (I) wherein R₂ is hydrogen; phenyl optionally substituted with nitro, hydroxy, halogen, amino, C₁₋₆ alkyl, C₂₋₁₀ acyl, C₂₋₁₀ alkyloxy; napthyl optionally substituted with nitro, hydroxy, halogen, amino, C₁₋₆ alkyl, C₂₋₁₀ acyl, C₂₋₁₀ alkyloxy; pyridyl; pyrimidinyl; thienyl; pyrazinyl; imidazoyl; pyrrolyl; indazolyl; or purinyl.

A further embodiment consists of a method of treatment of a viral infection in mammals, including humans, which include the step of administrating a therapeutically effective amount of compounds of formula (I) wherein R₁ and R₄ are hydrogen; R₅ is hydrogen or C₂₋₁₀ acyl; X is oxygen; Z is CH₂; and R₃ is selected from hydrogen, fluoro, bromo, chloro or iodo. Particularly when R₃ is hydrogen or fluoro.

A further embodiment consists of a method of treatment of a viral infection in mammals, including humans, which includes the step of the administration of a therapeutically effective amount of compounds of formula (I) wherein R₁ and R₄ are hydrogen; R₅ is hydrogen or C₂₋₁₀ acyl; X is oxygen; Z is CH₂; and R₅ is hydrogen; C₁₋₁₀ silylalkyl; C₂₋₁₀ acyloxy; phosphate; phosphonophosphate or phosphonodiphosphate.

An additional embodiment consists of a method of treatment of a viral infection in mammals, including humans, which includes the step of the administration of a therapeutically effective amount of compounds of formula (I) wherein R₁ and R₄ are hydrogen; R₅ is hydrogen or C₂₋₁₀ acyl; X is oxygen; Z is CH₂; and Y is oxygen.

A further embodiment consists of a method of treatment of a viral infection in mammals, including humans, which includes the step of the administration of a therapeutically effective amount of compounds of formula (I) wherein R₁ and R₄ are hydrogen; R₅ is hydrogen or C₂₋₁₀ acyl; X is oxygen; Z is CH₂; and Y is CH₂.

In an alternative aspect of the present invention includes a method for the treatment of a mammal, including humans, infected with or susceptible to a viral infection comprising the administration of an effective amount of a compound of formula (I) wherein X is oxygen; Y is oxygen or CH₂; R₁ and R₄ are hydrogen; R₃ is hydrogen orfluoro; R₂ is hydrogen, C₆₋₁₀ aryl, C₅₋₁₀ heterocycle, C₁₋₆ alkyl, C₂₋₆ acyloxycarbonyl or halogen; and R₅ is hydrogen, C₁₋₁₀ silylalkyl or C₂₋₁₀ acyl. Particularly when R₂ is hydrogen; phenyl optionally substituted with nitro, hydroxy, halogen, amino, C₁₋₆ alkyl, C₂₋₁₀ acyl, C₂₋₁₀ alkyloxy; napthyl optionally substituted with nitro, hydroxy, halogen, amino, C₁₋₆ alkyl, C₂₋₁₀ acyl, C₂₋₁₀ alkyloxy; pyridyl; pyrimidinyl; thienyl; pyrazinyl; imidazoyl; pyrrolyl; indazolyl; or purinyl.

In a further embodiment of the present invention is provided the use of a compound of formula (I) in the manufacture of a medicament for the treatment of human immunodeficiency virus.

In an alternative aspect of the present invention is provided a method for the treatment of a mammal, including human, infected with or susceptible to infection with the human immunodeficiency virus comprising the administration of an effective amount of a compound of formula (I).

It will be appreciated by people skilled in the art that treatment extend to prophylaxis as well to the treatment of established infections or symptoms.

The present invention includes the imidazopyrimidine nucleoside of formula (I)

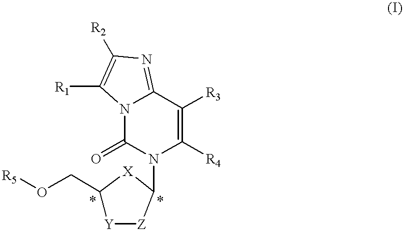

salts or esters of said compound, pharmaceutical acceptable derivatives or pharmaceutically acceptable salts or esters thereof, wherein:

X is oxygen;

Y is oxygen or CH₂;

R₁ and R₄ are hydrogen;

R₃ is hydrogen or fluoro;

R₂ is hydrogen, C₆₋₁₀ aryl, C₅₋₁₀ heterocycle, C₁₋₆ alkyl, C₂₋₆ acyloxycarbonyl or halogen; and

R₅ is hydrogen, C₁₋₁₀ silylalkyl or C₂₋₁₀ acyl.

Of particular interest are those compounds of formula (I) wherein R₂ is hydrogen, C₆₋₁₀ aryl or C₅₋₁₀ heterocycle. Specially those in which R₂ is hydrogen; phenyl optionally substituted with nitro, hydroxy, halogen, amino, C₁₋₆ alkyl, C₂₋₁₀ acyl, C₂₋₁₀ alkyloxy; napthyl optionally substituted with nitro, hydroxy, halogen, amino, C₁₋₆ alkyl, C₂₋₁₀ acyl, C₂₋₁₀ alkyloxy; pyridyl; pyrimidinyl; thienyl; pyrazinyl; imidazoyl; pyrrolyl; indazolyl; or purinyl.

The following compounds of formula (I) are also included in the present invention:

6-[(2S,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;

6-[(2R,5S)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;

6-[(2S,5R)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;

2-(4-NITROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;

8-FLUORO-2-(4-FLUOROPHENYL)-6-[(5R,2S)-5-HYDROXYMETHYL OXOLAN-2-YL]-5,6-DIHYDRO[1,3]DIAZOLO[1,2-C][1,3]DIAZIN-5-ONE;

8-FLUORO-2-(4-FLUOROPHENYL)-6-[(2R,5S)-5-HYDROXYMETHYL OXOLAN-2-YL]-5,6-DIHYDRO[1,3]-DIAZOLO[1,2-C][1,3]DIAZIN-5-ONE;

8-FLUORO-2-(4-NITROPHENYL)-6-[(5R,2S)-5-HYDROXYMETHYL OXOLAN-2-YL]-5,6-DIHYDRO[1,3]DIAZOLO[1,2-C][1,3]DIAZIN-5-ONE;

8-FLUORO-2-(4-NITROPHENYL)-6-[(2R,5S)-5-HYDROXYMETHYL OXOLAN-2-YL]-5,6-DIHYDRO[1,3]-DIAZOLO[1,2-C][1,3]DIAZIN-5-ONE;

2-(4-FLUOROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;

2-(4-NITROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;

6-[(2R,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;

8-FLUORO-2-(4-FLUOROPHENYL)-6-[(2S,5S)-5-HYDROXYMETHYL OXOLAN-2-YL]-5,6-DIHYDRO[1,3]-DIAZOLO[1,2-C][1,3]DIAZIN-5-ONE;

8-FLUORO-2-(4-NITROPHENYL)-[(2R,5R)-5-HYDROXYMETHYLOXOLAN-2-YL]-5,6-DIHYDRO[1,3]-DIAZOLO[1,2-C][1,3]DIAZIN-5-ONE;

8-FLUORO-2-(4-NITROPHENYL)-6-[(2S,5S)-5-HYDROXYMETHYL OXOLAN-2-YL]-5,6-DIHYDRO[1,3]-DIAZOLO[1,2-C][1,3]DIAZIN-5-ONE;

2-(4-FLUOROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;

2-(4-FLUOROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;

L2-(4-NITROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;

2-(4-NITROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;

8-FLUORO-2-(4-FLUOROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;

8-FLUORO-2-(4-FLUOROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;

8-FLUORO-2-(4-NITROPHENYL)-6-[(2S,4S)-5-HYDROXYMETHYL OXOLAN-4-YL]-5,6-DIHYDRO[1,3]-DIAZOLO[1,2-C][1,3]DIAZIN-5-ONE;

8-FLUORO-2-(4-NITROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;

2-(4-FLUOROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;

2-(4-FLUOROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;

2-(4-NITROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;

2-(4-NITROPHENYL)-6-[(2R,4S )-5-HYDROXYMETHYL-1,3-DIOXOLAN-4-YL]-5,6-DIHYDRO[1,3]-DIAZOLO[1,2-C][1,3]DIAZIN-5-ONE;

8-FLUORO-2-(4-FLUOROPHENYL)-6-[(2S,4R)-2-HYDROXYMETHYL-1,3-DIOXOLAN-4-YL]-5,6-DIHYDRO[1,3]DIOXOLO[1,2-C][1,3]DIAZIN-5-ONE;

8-FLUORO-2-(4-FLUOROPHENYL)-[(2R,4S)-5-HYDROXYMETHYL OXOLAN-4-YL]-5,6-DIHYDRO[1,3]-DIAZOLO[1,2-C][1,3]DIAZIN-5-ONE;

8-FLUORO-2-(4-NITROPHENYL)-6-[(2S,4R)-2-HYDROXYMETHYL-1,3-DIOXOLAN-4-YL]-5,6-DIHYDRO[1,3]DIAZOLO[1,2-C][1,3]DIAZIN-5-ONE;

8-FLUORO-2-(4-NITROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE;

4-(6-(2S,5R)-5-HYDROXYMETHYLOXOLAN-2-YL-(5-OXO-5,6-DIHYDRO[1,3-DIAZOLO[1,2-C][1,3]-DIAZIN-2-YL)PHENYLAMMONIUM CHLORIDE;

2-AMINO-3-METHYL-BUTYRIC ACID 4-[2-(4-FLUORO-PHENYL)-5-OXOIMIDAZO[1,2-C]PYRIMIDIN-6-YL]-[1,3]DIOXOLAN-2-YLMETHYL ESTER; HYDROCHLORIDE SALT;

6-(2-HYDROXYMETHYL-[1,3]DIOXOLAN-4-YL)-2-PYRIDIN-2-YL-6H-IMIDAZO[1,2-C]PYRIMIDIN-5-ONE; HYDROCHLORIDE SALT;

6-(2-HYDROXYMETHYL-[1,3]DIOXOLAN-4-YL)-2-PHENYL-6H-IMIDAZO[1,2-C]PYRIMIDIN-5-ONE;

6-(2-HYDROXYMETHYL-[1,3]DIOXOLAN-4-YL)-2-PYRIDIN-2-YL-6H-IMIDAZO[1,2-C]PYRIMIDIN-5-ONE;

6-(2-HYDROXYMETHYL-[1,3]DIOXOLAN-4-YL)-6H-IMIDAZO[1,2-C]PYRIMIDIN-5-ONE; and

6-(2-HYDROXYMETHYL-(1,3,)DIOXOLAN-4-YL)-3-PYRIDIN-3-YL-6H-IMIDAZO(1,2)PRIMIDIN-5-ONE.

An alternative embodiment of the present invention includes compounds of formula (I) wherein R₃ is halogen or hydrogen. Particularly those where R₃ is fluoro, bromo, iodo, chloro or hydrogen.

Compounds of formula (I) in which R₅ is hydrogen or C₂₋₁₀ acyl are within the scope of the present invention.

It will be appreciated by those skilled in the art that the compounds of formula (I) contain at least two chiral centres (shown as * in formula (I)) and thus exist in the form of two pairs of optical isomers (i.e. enantiomers) and mixtures thereof including racemic mixtures. Thus the compounds of formula (I) may be either cis isomers or trans isomers, or mixtures thereof. Each of the cis and trans isomers can exist as one of two enantiomers or as mixtures thereof including racemic mixtures. All such isomers and mixtures thereof including racemic mixtures are included within the scope of the invention.

By “a pharmaceutically acceptable salt or ester” is meant any pharmaceutically acceptable salt, ester, or salt of such ester, of compound I or any other compound which, upon administration to the recipient, is capable of providing (directly or indirectly) compound I or an antivirally active metabolite or residue thereof.

It will be appreciated by those skilled in the art that compound for formula (I) administered in the treatment object of the present invention may be modified to provide pharmaceutically acceptable derivatives thereof, at functional groups in both the base moiety and at the hydroxymethyl group of the sugar ring. Modification at all such functional groups are included within the scope of the invention. However of particular interest are pharmaceutically acceptable derivatives obtained by modification of the 2-hydroxymethyl group at 2-carbon of the sugar ring.

Preferred esters of compound of formula (I) include the compounds in which the hydrogen of the 2-hydroxymethyl group is replaced by an acyl function R—C(O)— in which the non-carbonyl moiety R of the ester is selected from hydrogen, straight or branched chain alkyl (e.g. methyl, ethyl, n-propyl, t-butyl, n-butyl), alkoxyalkyl (e.g. methoxymethyl), aralkyl (e.g. benzyl), aryloxyalkyl (e.g. phenoxymethyl), aryl (e.g. phenyl optionally substituted by halogen, C₁₋₄ alkyl or C₁₋₄ alkoxy); sulphonate esters such as alkyl- or aralkylsulphonyl (e.g. methanesulphonyl); amino acid esters (e.g. L-valyl or L-isoleucyl) and mono-, di- or tri-phosphate esters.

With regard to the above described esters, unless otherwise specified, any alkyl moiety present advantageously contains 1 to 16 carbon atoms, particularly 1 to 6 carbon atoms. Any aryl moiety present in such esters advantageously comprises a phenyl group.

In particular the esters may be a C₁₋₁₆ alkyl ester, an unsubstituted benzyl ester or a benzyl ester substituted by at least one halogen (bromine, chlorine, fluorine or iodine), C₁₋₆ alkyl, C₁₋₆ alkoxy, nitro or trifluoromethyl groups.

Pharmaceutically acceptable salts of the compound of formula (I) include those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, citric, methanesulphonic, formic, benzoic, malonic, naphthalene-2-sulphonic and benzenesulphonic acids. Other acids such as oxalic, while not in themselves pharmaceutically acceptable, may be useful as intermediates in obtaining the compounds of the invention and their pharmaceutically acceptable acid addition salts.

Salts derived from appropriate bases include alkali metal (e.g. sodium), alkaline earth metal (e.g. magnesium), ammonium and NR₄⁺ (where R is C₁₋₄ alkyl) salts.

The compounds of formula (I), either as an isononic mixture or as the individual enantiomer; and useful for the treatment of humans or mammalians to inhibit at least one of the following viruses: HCMV (Human Cytomegalovirus), HSV-1 or HSV-2 (Herpes Simplex 1 or 2), HIV (Human Immunodeficiency Virus), HTLV (Human T-lymphotropic virus), or HBV (Hepatitis B Virus). In a specific embodiment of the present invention compounds of formula (I) are active against HIV (Human Immunodeficiency Virus) and HBV (Hepatitis B Virus).

The nucleosides of formula (I) can be phosporylated to obtain a more active analogues. In order to facilitate the uptake of the phosphorylated nucleoside analogues and increase their bioavailability, several neutral monophosphorylated nucleoside prodrugs can be developed. These neutral nucleosides can be made more lipophilic due to the masking of the negative charge of the phosphate group with enzyme or pH labile neutral substitutes. This allows the analogue to penetrate the cell membrane much more readily than their corresponding 5′-monophosphate dianion counterpart. Once inside the cell, the analogue decomposes to generate the original monophosphorylated nucleoside analogue which can then be further phophorylated and incorporated into the viral DNA. To achieve this result several substituents can be used in the preparation of monophosphorylated nucleoside prodrugs. Examples of these substituents include S-acyl-2-thioethyls (SATE) (J. Med. Chem. (1995) 38:3941-3950, Antiviral Chem. Chemother. (1998) 9(1):41-52.) such as methyl (SATE), isopropyl (SATE), t-butyl (SATE) and phenyl (SATE), or carboxyloxymethyl such as pivaloyloxymethyl (POM) (Antiviral Chem. Chemother (1994) 5:91-98) and di-S-[(2-hydroxyethyl)sulfidyl]-2-thioethyl. Additionally, substituents such as alkyl methyl carbonates, for example isopropyl methyl carbonate (POC), can be used to form alkylmethyl carbamate prodrugs (Antiviral Chem. Chemother. (1997) 8: 557-564). A further option would be the synthesis of phenyl and benzylphosphotriesters analogues (Bioorg. Med. Chem. Lett. (1997) 7: 99-104) and phophostriesters analogues (WO98/17281) of nucleosides analogues can be prepared.

In a further aspect of the present invention, there is provided a method for the treatment of a viral infection in an infected host comprising the step of administering an antivirally effective dose of a compound of formula (I) as defined herein above or a pharmaceutically acceptable derivative thereof. In an alternative embodiment of the present invention the host is a mammal.

As will be appreciated by those skilled in the art, the compounds of formula (I) can be used prophylaxis as well as the treatment of established infections of symptoms.

The compounds of the present invention may also be useful in the treatment of AIDS-related conditions such as AIDS-related complex (ARC), persistent generalized lymphadenopathy (PGL), AIDS-related neurological conditions (such as dementia), anti-HIV antibody-positive and HIV-positive conditions, Kaposis sarcoma, thrombocytopenia purpura and opportunistic infections.

The compounds of the invention are also useful in the prevention or progression to clinical illness of individuals who are anti-HIV antibody or HIV-antigen positive and in prophylaxis following exposure to HIV.

The compounds of formula (I) or the pharmaceutically acceptable salts and esters thereof, may also be used for the prevention of viral contamination of biological fluids such as blood or semen in vitro.

The present invention also includes a commercial package containing a pharmaceutical agent comprising one or more compounds of formula (I) or the pharmaceutically acceptable salts or esters thereof. The pharmaceutical agent may further contain additional therapeutic agents therapeutically effective in the in vivo inhibition of HIV in mammals. The commercial package further includes instructions for the use of the pharmaceutical agent in the in vivo of inhibition of HIV in mammals. If required, the pharmaceutical agent is admixed with a pharmaceutically acceptable carrier, excipient or adjuvant. The pharmaceutical agent may be incorporated into a drug delivery device suitable for oral, rectal, nasal, topical (including buccal and sub-lingual), vaginal or parenteral (including intramuscular, sub-cutaneous and intravenous) administration or in a form suitable for administration by inhalation or insufflation and enclosed in a pharmaceutical acceptable container.

It will also be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, preferably in the range of 0.5 to 60 mg/kg/day, most preferably in the range of 1 to 20 mg/kg/day.

The desired dose may conveniently be presented in a single dose or as divided dose administered at appropriate intervals, for example as two, three, four or more sub-doses per day.

The compound of formula (1) is conveniently administered in unit dosage form; for example containing 10 to 1500 mg, conveniently 20 to 1000 mg, most conveniently 50 to 700 mg of active ingredient per unit dosage form.

Ideally the active ingredient should be administered to achieve peak plasma concentrations of the active compound of from about 1 to about 75 μM, preferably about 2 to 50 μM, most preferable about 3 to about 30 μM. This may be achieved, for example, by the intravenous injection of a 0.1 to 5% solution of the active ingredient, optionally in saline, or orally administered as a bolus containing about 1 to about 100 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

While it is possible that, for use in therapy, a compound of the invention may be administered as the raw chemical it is preferable to present the active ingredient as a pharmaceutical formulation. The invention thus further provides a pharmaceutical formulation comprising a compound of formula (I) or a pharmaceutically acceptable derivative thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical formulation suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds of formula (I) according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing an/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

For topical administration to the epidermis, the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Formulations suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents. Liquid sprays are conveniently delivered from pressurized packs.

For administration by inhalation the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired, the above described formulations may be adapted to give sustained release of the active ingredient. Such formulations are described in PCT publications WO 92/07555, WO 92/13521, WO 93/05843, WO 93/13756, WO 93/20138, WO 98/18452, WO 98/20930, WO 99/13864 WO 99/29297 and WO 00/23055,

The pharmaceutical compositions according to the invention may also contain other active ingredients such as antimicrobial, antifungal, and antiviral agents, immunomodulators or preservatives.

The compounds of the invention may also be used in combination with other therapeutic or prophylactic agents for example other antiinfective agents. In particular the compounds of the invention may be employed together with known antiviral, antimicrobial, or antifungal agents or immunomodulators. The invention thus provides, in a further aspect, a combination comprising a compound of formula (I) or) or a pharmaceutically acceptable derivative thereof together with another therapeutically active agent, in particular, an antiviral agent.

The therapeutically active agent to be used in combination with the compounds of the present invention may be selected from the group epivir, DAPD, FTC, AZT, d4T, nevirapine, DMP-226, nelfinavir, indinavir, delavirdine, MKC-442, 1592U89 (abacavir), 141W94, MK-639, saquinavir, ritonavir, TIBO, HEPT, BHAP, , α-APA, TSAO, calanolides, L-697,661, 2′,3′-dideoxycytidine (ddC), 2′,3′-dideoxyadenosine, 2′,3′-dideoxyinosine (ddl), 3′-deoxythymidine, 2′,3′-dideoxy-2′,3′-didehydro-thymidine, and 2∝,3′-dideoxy-2′,3′-didehydrocytidine and ribavirin; acyclic nucleosides such as acyclovir, ganciclovir, interferons such as alpha-, beta-and gamma-interferon; glucuronation inhibitors such as probenecid; nucleoside transport inhibitors such as dipyridamole; immunomodulators such as interleukin II (IL2) and granulocyte macrophage colony stimulating factor (GM-CSF), erythropoietin, ampligen, thymomodulin, thymopentin, foscarnet, glycosylation inhibitors such as 2-deoxy-D-glucose, castanospermine, 1-deoxynojirimycin; and inhibitors of HIV binding to CD4 receptors such as soluble CD4, CD4 fragments, CD4-hybrid molecules and inhibitors of the HIV aspartyl protease such as L-735,524.

In accordance with a further aspect of the present invention, the further therapeutic agent or agents may be chosen from epivir, DAPD, FTC, AZT, nevirapine, DMP-226, nelfinavir, indinavir, delavirdine, MKC-442, abacavir, 141W94, MK-639, saquinavir, ritonavir, acyclovir, interferon alpha, L-735,524, d4T, ddC, and ddl.

The individual components of such combinations may be administered either sequentially or simultaneously in separate or combined pharmaceutical formulations.

The combinations referred to above may conveniently be presented for use in the form of a pharmaceutical formulation and thus pharmaceutical formulations comprising a combination as defined above together with a pharmaceutically acceptable carrier thereof comprise a further aspect of the invention.

In another aspect method of treating a host infected with an HIV strain which includes administering an effective dose of a compound or the combinations of compounds of formula (I) capable of inhibiting viral replication.

When the compound of formula (I) or a pharmaceutically acceptable derivative thereof is used in combination with a second therapeutic agent active against the same virus, the dose of each compound may be either the same or differ from that when the compound is used alone. Appropriate doses will be readily appreciated by those skilled in the art.

The advantageous effects of the compounds of formula (I) and the second antiviral agents are observed over a wide ratio for example 1:250 to 250:1 alternatively 1:50 to 50:1, particularly about 1:10 to 10:1. Conveniently each compound will be employed in the combination in an amount at which it exhibits antiviral activity when used alone.

In an embodiment of the present invention the infected host is a mammal. Alternately, the infected host is human.

It is expected that the present combinations will be generally useful against viral infections or virus-associated tumors in humans, and the method of their use to inhibit viral infectivity or tumor growth in vitro or in vivo is also within the scope of the present invention.

Thus, there is provided, as a further aspect of the invention, a method for the treatment of a viral infection in a mammal, including man, comprising co-administration of an antiviral compound of formula (I) and a further antiviral report which inhibits HIV or HBV replication. Therapeutic methods comprising administration of a combination of a compound of formula (I) and more than one of the second antiviral agents, either together or in a plurality of paired combinations, is also within the scope of the invention.

It will be appreciated that the compound of formula (I) and the second antiviral agent may be administered either simultaneously, sequentially or in combination. If administration is sequential, the delay in administering the second of the active ingredients should not be such as to lose the benefit of the synergistic effect of the combination. Preferably administration will be simultaneous.

It will be appreciated by those skilled in the art that reference herein to treatment extends to prophylaxis as well as the treatment of established infections or symptoms.

It will be further appreciated that the amount of a combination of the invention required for use in treatment will vary not only with the particular compound selected but also with the route of administration, the nature of the condition being treated and the age and condition of the patient and will be ultimately at the discretion of the attendant physician or veterinarian. In general however a suitable dose will be in the range of from about 1 to about 750 mg/kg e.g. from about 10 to about 75-mg/kg of bodyweight per day, such as 3 to about 120 mg per kilogram body weight of the recipient per day, preferably in the range of 6 to 90 mg/kg/day, most preferably in the range of 15 to 60 mg/kg/day of each of the active ingredients of the combination.

The desired dose may conveniently be presented in a single dose or as divided doses administered at appropriate intervals, for example as two, three, four or more sub-doses per day.

While it is possible that, for use in therapy, a compound of the invention may be administered as the raw chemical it is preferable to present the active ingredient as a pharmaceutical formulation.

The invention thus further provides a pharmaceutical formulation comprising a compounds object of the present invention together with one or more pharmaceutically acceptable carriers thereof and, optionally, other therapeutic and/or prophylactic ingredients. The carrier(s) must be “acceptable” in the sense of being compatible with the other ingredients of the formulation and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical formulations suitable for oral administration may conveniently be presented as discrete units such as capsules, cachets or tablets each containing a predetermined amount of the active ingredient; as a powder or granules; as a solution; as a suspension; or as an emulsion. The active ingredient may also be presented as a bolus, electuary or paste. Tablets and capsules for oral administration may contain conventional excipients such as binding agents such as liquid glucose syrup acacia, gelatin, starch, mucilage, methylcellulose, polyvinylpyrrolidone, alginates, and pregelatinised starch; fillers for example lactose, microcrystalline cellulose, dicalcium phosphate, mannitol, magnesium carbonate, glycine, dextrose, sucrose, starch, mannitol, sorbitol and calcium carbonate; lubricants as for example stearic acid and magnesium stearate; disintegrants such as starch, alginic acid, microcrystalline cellulose, pectin, crossed-linked polyvinylpyrrolidone sodium starch glycollate and sodium carboxymethyl-cellulose; glidants for example talc and silica; preservatives for example sorbic acid and methyl or propyl parahydrobenzoate or pharmaceutically acceptable wetting agents such as sodium lauryl sulfate. Capsules will consist of a shell, normally of gelatin together with other ingredients such as glycerol, sorbitol, surface active agents, opaque fillers, preservatives, sweeteners, flavors and colors. The content of the capsule may further include diluents, lubricants and disintegrants The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils) or preservatives.

The compounds according to the present invention may also be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient or ingredients may be in powder form, obtained by aseptic isolation of sterile solid or by lyophilization from solution, for constitution with a suitable vehicle, e.g., sterile, pyrogen-free water, before use.

For topical administration to the epidermis, the compounds and combinations according to the invention may be formulated as ointments, creams or lotions, or as a transdermal patch. Ointments and creams may, for example, be formulated with an aqueous or oily base with the addition of suitable thickening and/or gelling agents. Lotions may be formulated with an lo aqueous or oily base and will in general also contain one or more emulsifying agents, stabilizing agents, dispersing agents, suspending agents, thickening agents, or coloring agents.

Formulations suitable for topical administration in the mouth include lozenges comprising active ingredient in a flavored based, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutically formulations suitable for rectal administration wherein the carrier is a solid, are most preferably represented as unit dose suppositories. Suitable carriers include cocoa butter and other materials commonly used in the art, and the suppositories may be conveniently formed by admixture of the active compound with the softened or melted carrier(s) followed by chilling and shaping in molds.

Formulations suitable for vaginal administration may be presented as pessaries, tampons, creams, gels, pastes, foams or sprays containing in addition to the active ingredient, such carriers as are known in the art to be appropriate.

For intra-nasal administration the compounds of the invention may be used as a liquid spray or dispersible powder or in the form of drops.

Drops may be formulated with an aqueous or non-aqueous base also comprising one or more dispersing agents, solubilizing agents or suspending agents. Liquid sprays are conveniently delivered from pressurized packs.

For administration by inhalation, the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired, the above described formulations adapted to give sustained release of the active ingredient, may be employed.

The pharmaceutical compositions according to the invention may also contain other active ingredients such as antimicrobial agents, or preservatives.

The compounds of the invention may also be used in combination with other therapeutic or prophylactic agents for example other antiinfective agents. In particular the compounds of the invention may be employed together with known antiviral, antimicrobial, or antifungal agents or immunomodulators. The invention thus provides, in a further aspect, a combination comprising a compound of formula (I) or a pharmaceutically acceptable derivative thereof together with another therapeutically active agent, in particular, an antiviral agent.

The therapeutically active agent to be used in combination with the compounds of the present invention may be selected from the group epivir, DAPD, FTC, AZT, d4T, nevirapine, DMP-226, nelfinavir, indinavir, delavirdine, MKC-442, 1592U89 (abacavir), 141W94, MK-639, saquinavir, ritonavir, TIBO, HEPT, BHAP, , α-APA, TSAO, calanolides, L-697,661, 2′,3′-dideoxycytidine (ddC), 2′,3′-dideoxyadenosine, 2′,3′-dideoxyinosine (ddl), 3′-deoxythymidine, 2′,3′-dideoxy-2′,3′-didehydro-thymidine, and 2′,3′-dideoxy-2′,3′-didehydrocytidine and ribavirin; acyclic nucleosides such as acyclovir, ganciclovir, interferons such as alpha-, beta-and gamma-interferon; glucuronation inhibitors such as probenecid; nucleoside transport inhibitors such as dipyridamole; immunomodulators such as interleukin II (IL2) and granulocyte macrophage colony stimulating factor (GM-CSF), erythropoietin, ampligen, thymomodulin, thymopentin, foscarnet, glycosylation inhibitors such as 2-deoxy-D-glucose, castanospermine, 1-deoxynojirimycin; and inhibitors of HIV binding to CD4 receptors such as soluble CD4, CD4 fragments, CD4-hybrid molecules and inhibitors of the HIV aspartyl protease such as L-735,524.

In an alternative embodiment of the present invention, the further therapeutic agent or agents may be chosen from epivir, DAPD, FTC, AZT, nevirapine, DMP-226, nelfinavir, indinavir, delavirdine, MKC-442, abacavir, 141W94, MK-639, saquinavir, ritonavir, acyclovir, interferon alfa, L-735,524, d4T, ddC, and ddl.

The individual components of such combinations may be administered either sequentially or simultaneously in separate or combined pharmaceutical formulations.

The combinations referred to above may conveniently be presented for use in the form of a pharmaceutical formulation and thus pharmaceutical formulations comprising a combination as defined above together with a pharmaceutically acceptable carrier thereof comprise a further aspect of the invention.

In another aspect method of treating a host infected with an HIV strain which includes administering an effective dose of a compound or the combinations of compounds of formula (I) capable of inhibiting viral replication.

When the compound of formula (I) or a pharmaceutically acceptable derivative thereof is used in combination with a second therapeutic agent active against the same virus, the dose of each compound may be either the same or differ from that when the compound is used alone. Appropriate doses will be readily appreciated by those skilled in the art.

The advantageous effects of the compounds of formula (I) and the second antiviral agents are observed over a wide ratio for example 1:250 to 250:1 alternatively 1:50 to 50:1, particularly about 1:10 to 10:1. Conveniently each compound will be employed in the combination in an amount at which it exhibits antiviral activity when used alone.

In an embodiment of the present invention the infected host is a mammal. Alternately, the infected host is human.

It is expected that the present combinations will be generally useful against viral infections or virus-associated tumours in humans, and the method of their use to inhibit viral infectivity or tumour growth in vitro or in vivo is also within the scope of the present invention.

Thus there is provided in a alternative aspect a method for the treatment of a viral infection in a mammal, including man, comprising co-administration of an antiviral compound of formula (I) and an inhibitor of HIV or HBV replication. Therapeutic methods comprising administration of a combination of a compound of formula (I) and more than one of the second antiviral agents, either together or in a plurality of paired combinations, is also within the scope of the invention.

It will be appreciated that the compound of formula (I) and the second antiviral agent may be administered either simultaneously, sequentially or in combination. If administration is sequential, the delay in administering the second of the active ingredients should not be such as to lose the benefit of the synergistic effect of the combination. Preferably administration will be simultaneous.

It will be appreciated by those skilled in the art that reference herein to treatment extends to prophylaxis as well as the treatment of established infections or symptoms.

It will be further appreciated that the amount of a combination of the invention required for use in treatment will vary not only with the particular compound selected but also with the route of administration, the nature of the condition being treated and the age and condition of the patient and will be ultimately at the discretion of the attendant physician or veterinarian. In general however a suitable dose will be in the range of from about 1 to about 750 mg/kg e.g. from about 10 to about 75-mg/kg of bodyweight per day, such as 3 to about 120 mg per kilogram body weight of the recipient per day, preferably in the range of 6 to 90 mg/kg/day, most preferably in the range of 15 to 60 mg/kg/day of each of the active ingredients of the combination.

The desired dose may conveniently be presented in a single dose or as divided doses administered at appropriate intervals, for example as two, three, four or more sub-doses per day.

Furthermore there is provided in this invention a process for synthesizing compound of formula (I).

The compounds of the invention can be synthesized as depicted in Schemes 1 or 2.

[chemical structure]

The various steps as illustrated in scheme 1 may be briefly described as follows.

### SCHEME 1

### Step 1

The compounds of formula (II) can be prepared by any method known in the art e.g. C. A. Evans et al. “Divergent asymmetric syntheses of dioxolane nucleoside analogues” Tetrahedron asymmetry, 4, pp 2319-2323 (1993) and U.S. Pat. No. 5,047,407.

The compounds of formula (Ill) can be prepared by any method known in the art e.g. G. Danswan et al. “Synthesis of (Imidazo[1,2-C]pyrimidin-2-yl) phenylmethanones and 6-Benzoylpyrrolo[2,3-d]pyrimidines” J. Heterocycl. Chem. 26, pp 293-299 (1989)

A previously silylated (or silylated in situ) (e.g. PCT publication WO92/20669) imidazopyrimidine base (III) is then glycosylated with a compound of formula (II) in the presence of a Lewis acid of formula (VIII) such as iodotrimethylsilane or trimethylsilyl triflate, to give a compound of formula (IV) as a mixture of cis and trans isomers.

Alternatively, the coupling can be achieved without the assistance of a Lewis acid. For example, in the Hibert-Johnson procedure, nucleosides can be synthesized by reacting a suitable base with a chlorosugar without the need of a Lewis acid as a catalyst. This procedure could also be suitable for preparing compounds of formula (IV).

### Step 2

The cis and trans compounds of formula (IV) are then separated by any method known in the art such as chromatography on silica gel, fractionnal recrystallisation, or by HPLC techniques. Each pure isomers is then deprotected by any method known in the art to afford compounds of formula (I).

Alternatively, the compounds of formula (IV) are deprotected by any method known in the art and the isomers are then separated to give the isomers of formula (I)

[chemical structure]

The various steps as illustrated in scheme 2 may be briefly described as follows.

### SCHEME 2

### Step 1

The compounds of formula (II) can be prepared by any method known in the art e.g. C. A Evans et al. “Divergent asymmetric syntheses of dioxolane nucleoside analogues”, Tetrahedron asymmetry, 4, pp 2319-2323 (1993).

A previously silylated (or silylated in situ) pyrimidine base or analogue thereof is then glycosylated with a compound of formula (II) in the presence of a Lewis acid of formula (VIII) to give a compound of formula (VI) either as a single isomer e.g. W-B Choi et al. “In situ complexation directs the stereochemistry of N-glycosylated in the synthesis of oxathiolanyl and dioxolanyl nucleoside analogues” J. Am. Chem. Soc. 113, 9377-9379 (1991) or as a mixture of the two isomers e.g. D. C Humber et al. “Expeditious preparation of (−)-2′-Deoxy-3′thiacytidine (3TC)” Tetrahedron letters, 32, pp 4625-4628 (1992).

As mentioned earlier, the coupling can be achieved without the assistance of a Lewis acid. For example, in the Hibert-Johnson procedure, nucleosides can be synthesized by reacting a suitable base with a chlorosugar without the need of a Lewis acid as a catalyst. This procedure could also be suitable for preparing compounds of formula (IV).

### Step 2

If necessary, the cis and trans compounds of formula (VI) are then separated by any method known in the art such as chromatography on silica gel, fractionnal recrystallisation, or by HPLC techniques. Each pure isomer is then deprotected by any method known in the art to afford compounds of formula (I).

Alternatively, the compounds of formula (VI) are deprotected by any method known in the art and the isomers are then separated to give the isomers of formula (VII)

### Step 3

The compounds of formula (VII) are condensed with an activated halogen intermediate such as an α-halocarbonyl compound of formula (IX) or 1-halooxiranes or mucochloric acid in the presence of an appropriate solvent such as methanol to give a compound of formula (I).

As used in this application, “P” is an alcohol protecting group such as acyl, aryl or silyl.

As used in this application, “W”, is an halogen.

As used in this application, “L” is a “leaving group”, i.e., an atom or a group which is displaceable upon reaction with an appropriate immidazopyrimidine or pyrimidine base or analogue thereof, with or without the presence of a Lewis acid. Suitable leaving groups include acyloxy groups, alkoxy groups, e.g., alkoxy carbonyl groups such as ethoxy carbonyl; halogens such as fluorine; amido; azido; isocyanato; substituted or unsubstituted, saturated or unsaturated thiolates, such as thiomethyl or thiophenyl; substituted or unsubstituted, saturated or unsaturated seleno, seleninyl, or selenoyl compounds, such as phenyl selenide or alkyl selenide.

A suitable leaving group may also be —OR_{L}, wherein R_{L} is a substituted or unsubstituted, saturated or unsaturated alkyl group, e.g. C₁₋₆ alkyl or alkenyl group; a substituted or unsubstituted aliphatic or aromatic acyl group e.g. C₁₋₆ aliphatic acyl group such as acetyl; a substituted or unsubstituted aromatic acyl group such as benzoyl; a substituted or unsubstituted, saturated or unsaturated alkoxy or aryloxy carbonyl group, such as methyl carbonate and phenyl carbonate; substituted or unsubstituted sulphonyl imidazolide; substituted or unsubstituted aliphatic or aromatic amino carbonyl group, such as phenyl carbamate; substituted or unsustituted alkyl imidiate group such as trichloroacetamidate; substituted or unsubstituted , saturated or unsaturated phosphonate, such as diethylphosphate; substituted or unsubstituted aliphatic or aromatic sulphinyl or sulphonyl group, such as tosylate; or hydrogen.

The Lewis acids used in the process of the preparation of the compounds of formula (I) have the general formula (VIII)

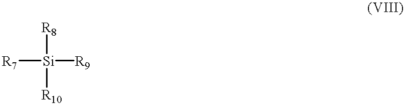

wherein R₇, R₈ and R₉ are independently selected from the group consisting of hydrogen, C₁₋₂₀ alkyl (e.g., methyl, ethyl, t-butyl), optionally substituted by halogens (F, Cl, Br, I), C₆₋₂₀alkoxy (e.g., methoxy) or C₆₋₂₀ aryloxy (e.g., phenoxy); C₇₋₂₀ aralkyl (e.g., benzyl), optionally substituted by halogen, C₁₋₂₀ alkyl or C₁₋₂₀ alkoxy (e.g., p-methoxybenzyl); C₆₋₂₀ aryl (e.g., phenyl), optionally substituted by halogens C₁₋₂₀ alkyl or C₁₋₂₀ alkoxy; trialkylsilyl; halogens (F, Cl, Br, I).

R₁₀ is selected from the group consisting of halogen (F, Cl, Br, I); C₁₋₂₀ sulphonate esters optionally substituted by halogens (e.g., trifluoromethane sulphonate); C₁₋₂₀ alkyl esters, optionally substituted by halogen (e.g., triluoroacetate); polyvalent halides (e.g., triiodide); trisustituted silyl groups of the general formula (R₇)(R₈)(R₉)Si (wherein R₇, R₈ and R₉ are as defined above); saturated or unsaturated selenyl C₆₋₂₀ aryl; substituted or unsubstituted C₆₋₂₀ arylsulphenyl; substituted or unsubstituted C₁₋₂₀ alkoxyalkyl; and trialkylsiloxy.

Specific compounds of formula (I) include:

2-HYDROXYMETHYL-6-[CIS-2-HYDROXYMETHYL-5′-(1′,3′-OXATHIOLANYL)]-IMIDAZO(1,2-C)PYRIMIDIN-5(6H)-ONE (COMPOUND #1);

2-PHENYL-6-[CIS-2′-HYDROXYMETHYL-5′-(1′,3′-OXATHIOLANYL)]-IMIDAZO(1,2-C)PYRIMIDIN-5(6H)-ONE (COMPOUND #2);

2-(4″-NITROPHENYL)-6-[CIS-2′-HYDROXYMETHYL-5′-(1′,3′-OXATHIOLANYL)]-IMIDAZO(1,2-C)PYRIMIDIN-5(6H)-ONE (COMPOUND #3);

2-(4′-METHOXYPHENYL)-6-[CIS-2′-HYDROXYMETHYL-5′-(1′,3′-OXATHIOLANYL)]IMIDAZO(1,2-C)PYRIMIDIN-5(6H)-ONE (COMPOUND #4);

2′-(2-NAPHTHYL)-6-(CIS-2′-HYDROXYMETHYL-5′-(1′,3′-OXATHIOLANYL))-IMIDAZO(1,2-C)PYRIMIDIN-5(6H)-ONE (COMPOUND #5);

2-METHYL-6-[CIS-2′-HYDROXYMETHYL-5′-(1′,3′-OXATHIOLANYL)]-IMIDAZO(1,2-C)PYRIMIDIN-5(6H)-ONE (COMPOUND #6);

2-(4″-FLUOROPHENYL)-6-CIS[2′-HYDROXYMETHYL-5′-(1′,3′-OXATHIOLANYL)]-IMIDAZO(1,2-C)PYRIMIDIN-5(6H)-ONE (COMPOUND #7);

CIS-6-[2′-HYDROXYMETHYL-5′-(1′,3′-OXATHIOLANYL)]-IMIDAZO(1,2-C)PYRIMIDIN-5(6H)-ONE (COMPOUND #8);

2-HYDROXYMETHYL-6-(TRANS-2′-HYDROXYMETHYL-5′-(1′,3′-OXATHIOLANYL))-IMIDAZO(1,2-C) PYRIMIDIN-5(6H)-ONE (COMPOUND #9);

ETHYL-6-[2′R-HYDROXYMETHYL-5′S-(1′,3′-OXATHIOLANYL)]-IMIDAZO(1,2-C)PYRIMIDIN-5(6H)-ONE-2-CARBOXYLATE (COMPOUND #10);

(−)-2-HYDROXYMETHYL-6-(-2′R-HYDROXYMETHYL-5′S-(1′,3′-OXATHIOLANYL))-IMIDAZO(1,2-C)PYRIMIDIN-5(6H)-ONE (COMPOUND #11);

2-PHENYL-6-(2′R-HYDROXYMETHYL-5′S-(1′,3′-OXATHIOLANYL)-IMIDAZO(1,2-C)PYRIMIDIN-5(6H)-ONE (COMPOUND #12);

2-(4″-NITROPHENYL-6-(2′R-HYDROXYMETHYL-5′S-(1′,3′-OXATHIOLANYL)-IMIDAZO(1,2-C)PYRIMIDIN-5(6H)-ONE (COMPOUND #13);

2-(4″-FLUOROPHENYL-6-(2′R-HYDROXYMETHYL-5′S-(1′,3′-OXATHIOLANYL)-IMIDAZO(1,2-C)PYRIMIDIN-5(6H)-ONE (COMPOUND #14);

(−)-2-HYDROXYMETHYL-6-(-2′S-HYDROXYMETHYL-5′R-(1′,3′-OXATHIOLANYL))-IMIDAZO(1,2-C)PYRIMIDIN-5(6H)-ONE (COMPOUND #15);

CIS-2-HYDROXYMETHYL-5-((N-6′-5′-6′-DIHYDRO-2′-CARBOETHOXY-5′-OXOIMIDAZO(1,2-C)PYRIMIDINE)-1,3-OXATHIOLANE (COMPOUND #16);

2-(4″-FLUOROPHENYL)6-[CIS-2′-HYDROXYMETHYL-5′-(1′,3′-DIOXOLANYL)]-IMIDAZO(1,2-C)PYRIMIDIN-5(6H)-ONE (COMPOUND #17);

2-HYDROXYMETHYL-6-(CIS-2′-HYDROXYMETHYL-5′-(1′,3′-DIOXOLANYL))-IMIDAZO(1,2-C)PYRIMIDIN(6H)-ONE (COMPOUND #18);

2-METHYLCARBOXYLATE-6-(CIS-2′-HYDROXYMETHYL-5′-(1,3-DIOXOLANYL))-IMIDAZO(1,2-C)PYRIMIDIN5(6H)-ONE (COMPOUND #19);

2-HYDROXYMETHYL-6-(CIS-2′-HYDROXYMETHYL-5′-(1′,3′-DIOXOLANYL)-8-FLUOROIMIDAZO(1,2-C)PYMIMIDIN5(6H)-ONE (COMPOUND #20);

2-(4″-NITROPHENYL)-6-(CIS-2′-HYDROXYMETHYL-5′-(1′,3′-DIOXOLANYL))-IMIDAZO(1,2-C)PYRIMIDIN(6H)-ONE (COMPOUND #21);

2-(4″-NITROPHENYL)-6-(CIS-2′-HYDROXYMETHYL-5′-(1′,3′-DIOXOLANYL)-8-FLUOROIMIDAZO(1,2-C)PYRIMIDIN5(6H)-ONE (COMPOUND #22);

2-HYDROXYMETHYL-6-(TRANS-2′-HYDROXYMETHYL-5′-(1′,3′-DIOXOLANYL)-8-FLUOROIMIDAZO(1,2-C)PYMIMIDIN5(6H)-ONE (COMPOUND #23);

2-METHYLCARBOXYLATE-6-(TRANS-2′-HYDROXYMETHYL-5′-(1′,3′-DIOXOLANYL))-IMIDAZO(1,2-C)PYRIMIDIN5(6H)-ONE (COMPOUND #24);

2-HYDROXYMETHYL-6-(TRANS-2′-HYDROXYMETHYL-5′-(1′,3′-DIOXOLANYL))-IMIDAZO(1,2-C)PYRIMIDIN5(6H)-ONE (COMPOUND #25);

2-(4″NITROPHENYL)-6-(TRANS-2′-HYDROXYMETHYL-5′-(1′,3′-DIOXOLANYL))-IMIDAZO(1,2-C)PYRIMIDIN5(6H)-ONE (COMPOUND #26);

2-(4″-NITROPHENYL)-6-(TRANS-2′-HYDROXYMETHYL-5′-(1′,3′-DIOXOLANYL)-8-FLUOROIMIDAZO(1,2-C)PYRIMIDIN5(6H)-ONE (COMPOUND #27); AND

2-(4″-NITROPHENYL)-6-(2′S-HYDROXYMETHYL-5′R-(TETRAHYDROFURANYL))-IMIDAZO(1,2-C)PYRIMIDIN5(6H)-ONE(COMPOUND #28).

Additionally, compounds of the present invention include: Compound No.:

| Compound | | |
|---|---|---|
| No.: | | |
| 29 | 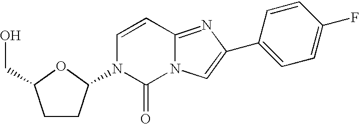 | 6-[(2S,5R)-5-HYDROXYMETHYL OXOLAN-2-YL]-2-(4-FLUORO PHENYL)- 5,6-DIHYDRO[1,3]-DIAZOLO[1,2-C][1,3]DIAZIN-5-ONE |
| 30 | 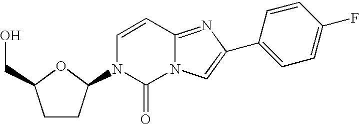 | 6-[(2R,5S)-5-HYDROXYMETHYL OXOLAN-2-YL]-2-(4-NITROPHENYL)-5,6- DIHYDRO[1,3]-DIAZOLO[1,2-C][1,3]DIAZIN-5-ONE |
| 31 | 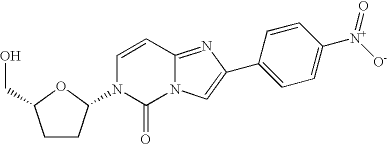 | 6-[(2S,5R)-5-HYDROXYMETHYL OXOLAN-2-YL]-2-(4-NITROPHENYL)-5,6- DIHYDRO[1,3]-DIAZOLO[1,2-C][1,3]DIAZIN-5-ONE |
| 32 | 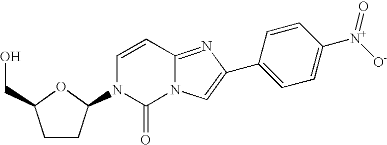 | 2-(4- NITROPHENYL)-6-[(2R,5S)-5- HYDROXYMETHYLOXOLAN-2-YL]-5,6- DIHYDRO[1,3]-DIAZOLO[1,2-C][1,3]DIAZIN-5-ONE |
| 33 | 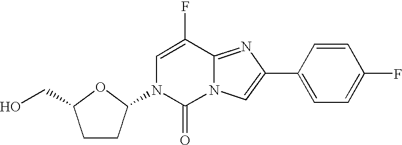 | 8-FLUORO-2-(4-FLUOROPHENYL)-6- [(5R,2S)-5-HYDROXYMETHYL OXOLAN- 2-YL]-5,6-DIHYDRO[1,3]DIAZOLO[1,2- C][1,3]DIAZIN-5-ONE |
| 34 | 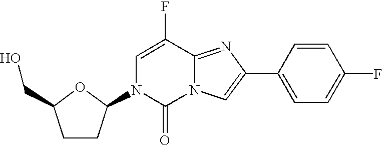 | 8-FLUORO-2-(4-FLUOROPHENYL)-6- [(2R,5S)-5-HYDROXYMETHYL OXOLAN- 2-YL]-5,6-DIHYDRO[1,3]-DIAZOLO[1,2- C][1,3]DIAZIN-5-ONE |
| 35 | 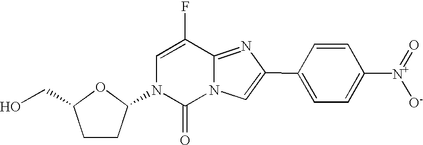 | 8-FLUORO-2-(4-NITROPHENYL)-6- [(2R,5S)-5-HYDROXYMETHYL OXOLAN- 2-YL]-5,6-DIHYDRO [1,3]DIAZOLO[1,2- C][1,3]DIAZIN-5-ONE |
| 36 | 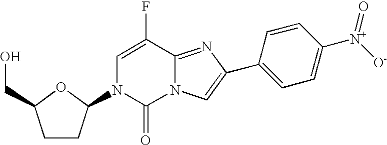 | 8-FLUORO-2-(4-NITROPHENYL)-6- [(2R,5S)-5-HYDROXYMETHYL OXOLAN- 2-YL]-5,6-DIHYDRO[1,3]-DIAZOLO[1,2- C][1,3]DIAZIN-5-ONE |
| 37 | 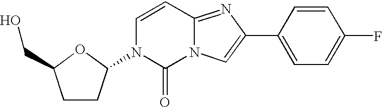 | 2-(4-FLUOROPHENYL)-6-[(2S,5S)-5- HYDROXYMETHYLOXOLAN-2-YL]-5,6- DIHYDRO[1,3]-DIAZOLO[1,2-C][1,3]DIAZIN-5-ONE |
| 38 | 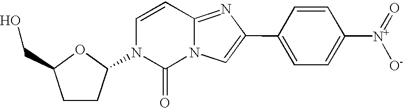 | 2-(4-NITROPHENYL)-6-[(2S,5S)-5- HYDROXYMETHYLOXOLAN-2-YL]-5,6- DIHYDRO[1,3]-DIAZOLO[1,2-C][1,3]DIAZIN-5-ONE |
| 39 | 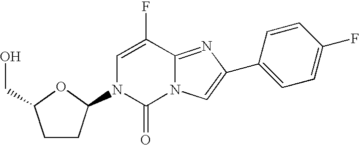 | 6-[(2R,5R)-5-HYDROXYMETHYL OXOLAN-2-YL]-2-(4-FLUORO PHENYL)- 5,6-DIHYDRO[1,3]-DIAZOLO[1,2-C][1,3]DIAZIN-5-ONE |
| 40 | 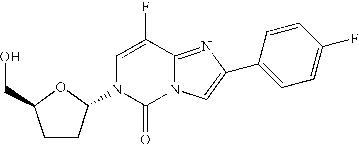 | 8-FLUORO-2-(4-FLUOROPHENYL)-6- [(2S,5S)-5-HYDROXYMETHYL OXOLAN-2- YL]-5,6-DIHYDRO[1,3]-DIAZOLO[1,2- C][1,3]DIAZIN-5-ONE |
| 41 | 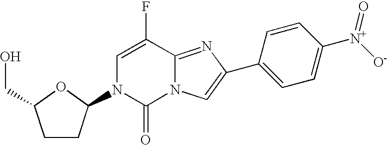 | 8-FLUORO-2-(4-NITROPHENYL)- [(2R,5R)-5-HYDROXYMETHYL OXOLAN- 2-YL]-5,6-DIHYDRO[1,3]-DIAZOLO[1,2- C][1,3]DIAZIN-5-ONE |
| 42 | 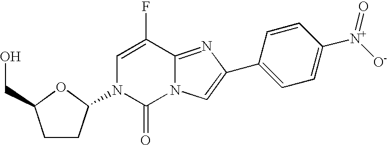 | 8-FLUORO-2-(4-NITROPHENYL)-6- [(2S,5S)-5-HYDROXYMETHYL OXOLAN-2- YL]-5,6-DIHYDRO[1,3]-DIAZOLO[1,2- C][1,3]DIAZIN-5-ONE |
| 43 | 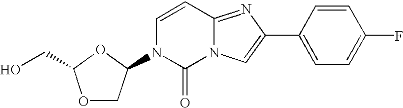 | 2-(4-FLUOROPHENYL)-6-[(2S,4S)-2- HYDROXYMETHYL-1,3-DIOXOLAN-4-YL]- 5,6-DIHYDRO[1,3]DIAZOLO[1,2- C][1,3]DIAZIN-5-ONE |
| 44 | 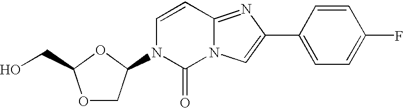 | 2-(4-FLUOROPHENYL)-6-[(2R,4R)-2- HYDROXYMETHYL-1,3-DIOXOLAN-4-YL]- 5,6-DIHYDRO[1,3]DIAZOLO[1,2- C][1,3]DIAZIN-5-ONE |
| 45 | 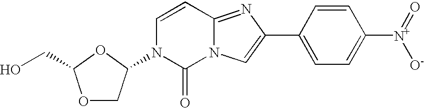 | L2-(4-NITROPHENYL)-6-[(2S,4S)-2- HYDROXYMETHYL-1,3-DIOXOLAN-4-YL]- 5,6-DIHYDRO[1,3]DIAZOLO[1,2- C][1,3]DIAZIN-5-ONE |
| 46 | 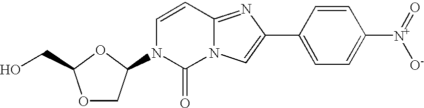 | 2-(4-NITROPHENYL)-6-[(2R,4R)-2- HYDROXYMETHYL-1,3-DIOXOLAN-4-YL]- 5,6-DIHYDRO[1,3]DIAZOLO[1,2- C][1,3]DIAZIN-5-ONE |
| 47 | 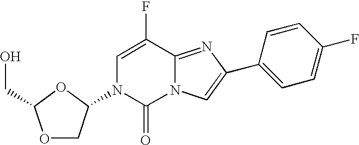 | 8-FLUORO-2-(4-FLUOROPHENYL)-6- [(2S,4S)-5-HYDROXYMETHYL OXOLAN-4- YL]-5,6-DIHYDRO[1,3]-DIAZOLO[1,2- C][1,3]DIAZIN-5-ONE |
| 48 | 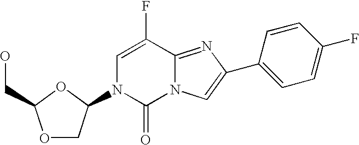 | 8-FLUORO-2-(4-FLUOROPHENYL)- [(2R,4R)-2-HYDROXYMETHYL-1,3- DIOXOLAN-4-YL]-5,6-DIHYDRO[1,3]- DIAZOLO[1,2-C][1,3]DIAZIN-5-ONE |
| 49 | 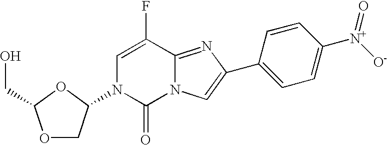 | 8-FLUORO-2-(4-NITROPHENYL)-6- [(2S,4S)-5-HYDROXYMETHYL OXOLAN-4- YL]-5,6-DIHYDRO[1,3]-DIAZOLO[1,2- C][1,3]DIAZIN-5-ONE |
| 50 | 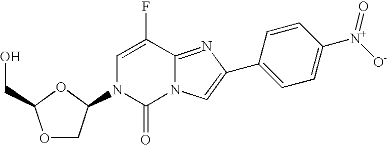 | 8-FLUORO-2-(4-NITROPHENYL)- [(2R,4R)-2-HYDROXYMETHYL-1,3- DIOXOLAN-4-YL]-5,6-DIHYDRO[1,3]- DIAZOLO[1,2-C][1,3]DIAZIN-5-ONE |
| 51 | 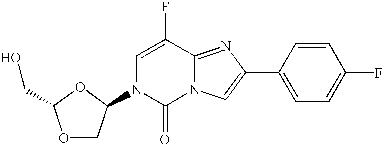 | 2-(4-FLUOROPHENYL)-6-[(2S,4R)-5- HYDROXYMETHYL-1,3-DIOXOLAN-4-YL]- 5,6-DIHYDRO[1,3]-DIAZOLO[1,2-C][1,3]DIAZIN-5-ONE |
| 52 | 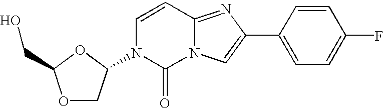 | 2-(4-FLUOROPHENYL)-6-[(2R,4S)-5- HYDROXYMETHYL-1,3-DIOXOLAN-4-YL]- 5,6-DIHYDRO[1,3]-DIAZOLO[1,2-C][1,3]DIAZIN-5-ONE |
| 53 | 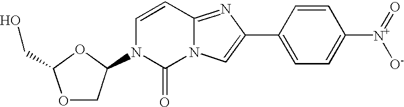 | 2-(4-NITROPHENYL)-6-[(2S,4R)-5- HYDROXYMETHYL-1,3-DIOXOLAN-4-YL]- 5,6-DIHYDRO[1,3]-DIAZOLO[1,2-C][1,3]DIAZIN-5-ONE |
| 54 | 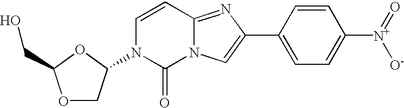 | 2-(4-NITROPHENYL)-6-[(2R,4S)-5- HYDROXYMETHYL-1,3-DIOXOLAN-4-YL]- 5,6-DIHYDRO[1,3]-DIAZOLO[1,2-C][1,3]DIAZIN-5-ONE |
| 55 | 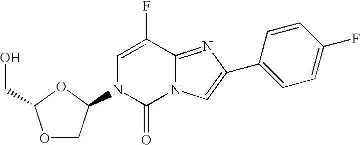 | 8-FLUORO-2-(4-FLUOROPHENYL)-6- [(2S,4R)-2-HYDROXYMETHYL-1,3- DIOXOLAN-4-YL]-5,6- DIHYDRO[1,3]DIOXOLO[1,2- C][1,3]DIAZIN-5-ONE |
| 56 | 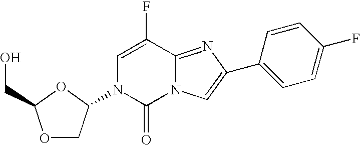 | 8-FLUORO-2-(4-FLUOROPHENYL) [(2R,4S)-5-HYDROXYMETHYL OXOLAN- 4-YL]-5,6-DIHYDRO[1,3]-DIAZOLO[1,2- C][1,3]DIAZIN-5-ONE |
| 57 | 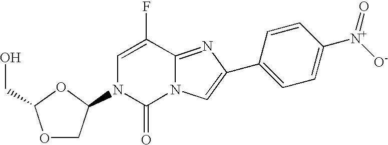 | 8-FLUORO-2-(4-NITROPHENYL)-6- [(2S,4R)-2-HYDROXYMETHYL-1,3- DIOXOLAN-4-YL]-5,6-DIHYDRO [1,3]DIAZOLO[1,2-C][1,3]DIAZIN-5-ONE |
| 58 | 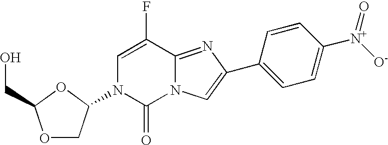 | 8-FLUORO-2-(4-NITROPHENYL)- [(2R,4S)-5-HYDROXYMETHYL OXOLAN- 4-YL]-5,6-DIHYDRO[1,3]-DIAZOLO[1,2- C][1,3]DIAZIN-5-ONE |
| 59 | 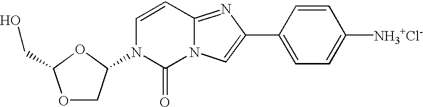 | 4-(6-(2S,5R)-5-HYDROXYMETHYL OXOLAN-2-YL-(5-OXO-5,6-DIHYDRO[1,3- DIAZOLO[1,2-C][1,3]-DIAZIN-2- YL)PHENYLAMMONIUM CHLORIDE |
| 60 | [chemical structure] | 2-AMINO-3-METHYL-BUTYRIC ACID 4-[2- (4-FLUORO-PHENYL)-5-OXO-IMIDAZO [1,2-C]PYRIMIDIN-6-YL]-[1,3]DIOXOLAN-2- YLMETHYL ESTER; HYDROCHLORIDE SALT |
| 61 | 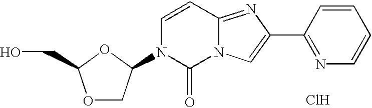 | 6-(2-HYDROXYMETHYL-[1,3]DIOXOLAN- 4-YL)-2-PYRIDIN-2-YL-6H-IMIDAZO[1,2-C]PYRIMIDIN-5-ONE; HYDROCHLORIDE SALT |
| 62 | 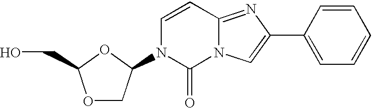 | 6-(2-HYDROXYMETHYL-[1,3]DIOXOLAN- 4-YL)-2-PHENYL-6H-IMIDAZO[1,2-C]PYRIMIDIN-5-ONE |
| 63 | 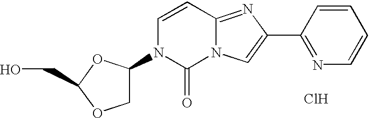 | 6-(2-HYDROXYMETHYL-[1,3]DIOXOLAN- 4-YL)-2-PYRIDIN-2-YL-6H-IMIDAZO[1,2- C]PYRIMIDIN-5-ONE |
| 64 | 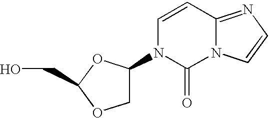 | 6-(2-HYDROXYMETHYL-[1,3]DIOXOLAN- 4-YL)-6H-IMIDAZO[1,2-C]PYRIMIDIN-5- ONE |
| 65 | [chemical structure] | 6-(2-HYDROXYMETHYL-(1,3,) DIOXOLAN-4-YL)-3-PYRIDIN-3-YL-6H- IMIDAZO(1,2)PRIMIDIN-5-ONE |

The scope of the present invention includes the following compounds:

2-(4-FLUOROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;

2-(4-FLUOROPHENYL)-6-[(2 R,4R)-2-HYDROXYMETHYL-1,3-DIOXOLAN-4-YL]-5,6-DIHYDRO[1,3]DIAZOLO[1,2-C][1,3]DIAZIN-5-ONE;

8-FLUORO-2-(4-FLUOROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE; and

8-FLUORO-2-(4-NITROPHENYL)-6-[(2S,4S)-5-HYDROXYMETHYLOXOLAN-4-YL]-5,6-DIHYDRO[1,3]-DIAZOLO[1,2-C].

The compounds, processes and method of treatment, object of the present invention are illustrated by the following examples which should not be interpreted as a limitation thereof.

### EXAMPLE 1

### ETHYL-6-[CIS-2′-HYDROXYMETHYL-5′-(1′,3′-OXATHIOLANYL)]-IMIDAZO(1,2-C)PYRIMIDIN-5(6H)-ONE-2-CARBOXYLATE

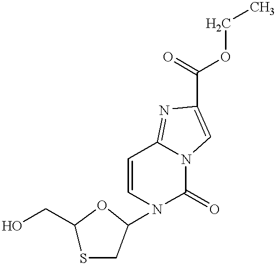

To a 100 ml flask equipped with a condenser were added ethyl bromopyruvate (1.15 ml, 1.70 g, 8.72 mmol), (+/−)-2′-deoxy-3′-thiacytidine (0.500 g, 2.18 mmol), and methanol (40 ml). The reaction was refluxed for 20 hours, cooled and diluted with H₂O (50 ml). After removing the methanol, the remaining aqueous layer was extracted with dichloromethane (3×), dried over MgSO₄, and adsorbed directly on silica gel. After purification by column chromatography (eluting with ethyl acetate) 54 mg of the desired product and 192 mg of the ethyl ester containing ca. 10% of the corresponding methyl ester were isolated (35%) as a white solid:

m.p. 154-156° C.;

¹H NMR (CDCl₃) δ 1.41 (t, 3H, J=7.1 Hz), 3.18 (dd, 1H, J=5.5, 11.8 Hz), 3.53 (dd, 1H, J=5.6, 12.3 Hz), 3.94 (dd, H, J=3.8, 12.6 Hz), 4.10 (dd, 1H, J=2.5, 12.0 Hz), 4.42 (q, 2H, J=7.2 Hz), 5.36 (t, 1H, J=2.7 Hz), 6.54 (t, 1H, J=5.5 Hz), 6.61 (d, 1H, J=8.0 Hz), 7.66 (d, 1H, J=8.0 Hz), 8.31 (s, 1H);

¹³C NMR (CDCl₃) δ 14.9, 37.3, 61.2, 66.7, 86.5, 87.3, 99.5, 118.0, 129.0, 136.8, 145.3, 145.5, 162.4.

### EXAMPLE 2

### 2-HYDROXYMETHYL-6-(CIS-2′-HYDROXYMETHYL-5′-[1′,3′-OXATHIOLANYL)]-IMIDAZO(1,2-C)PYRIMIDIN-5(6H)-ONE (compound #1)

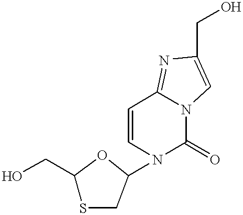

To a slurry of lithium aluminum hydride (23 mg, 0.609 mmol) in THF (20 ml) at 0° C. was added a solution of ethyl-6-[cis-2′-hydroxymethyl-5′-(1′,3′-oxathiolanyl)]-imidazo(1,2-C)pyrimidin-5(6H )-one-2-carboxylate (example 1) (0.198 g, 0.609 mmol) in THF (10 ml). The reaction mixture was heated for 20 hours and then quenched with 0.5 ml of saturated ammonium chloride solution. The solution was concentrated and the resulting residue was dissolved in dichloromethane (50 ml), washed with brine, dried over MgSO₄, and adsorbed directly on silica gel. The reaction mixture was purified by column chromatography (eluting with 5% methanol/dichloromethane) to yield 25 mg of product (13%) isolated as a white solid:

m.p. 60-64° C.;

¹H NMR (CD₃OD) δ 3.08 (dd, 1H, J=4.6, 11.8 Hz), 3.37 (dd, 1H, J=5.6, 12.0 Hz), 3.67 (dd, 1H, J=4.4, 12.4 Hz), 3.75 (dd, 1H, J=3.7, 12.4 Hz) 4.43 (s, 2H), 5.12 (t, 1H, J=3.7 Hz), 6.34 (t, 1H, J=5.5 Hz), 6.39 (d, 1H, J=8.0 Hz), 7.45 (s, 1H), 7.70 (d, 1H, J=8.0 Hz);

¹³C NMR (DMSO) δ 37.8, 59.0, 64.3, 87.9, 88.7, 98.6, 110.7, 130.4, 146.7, 146.9, 147.3.

### EXAMPLE 3

### PHENYL-6-[CIS-2′-HYDROXYMETHYL-5′-(1′,3′-OXATHIOLANYL)]-IMIDAZO(1,2-C)PYRIMIDIN-5(6H)-ONE (compound #2)

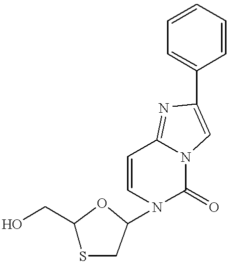

To a 50 ml flask equipped with a condenser were added α-bromoacetophenone (0.200 g, 1.00 mmol), (+/−)-2′-deoxy-3′-thiacytidine (0.100 g, 0.44 mmol), and methanol (20 ml). The reaction was refluxed for 18 hours. The cooled reaction was diluted with H₂O (20 ml) and the methanol was removed in vacuo. After extraction of the aqueous layer with dichloromethane (3×), the combined organic layers were dried over MgSO₄, concentrated and triturated with ethyl ether to yield 45 mg (31%) of the desired product as a white solid.:

m.p. 163-164° C.;

¹H NMR (DMSO) δ 3.40 (dd, 1H, J=3.6, 12.1 Hz), 3.62 (dd, 1H, J=5.8, 12.2 Hz), 3.80 (d, 2H, J=4.1 Hz), 5.32 (t, 1H, J=3.9 Hz), 5.77 (s, 1H), 6.50 (t, 1H, J=5.4 Hz), 6.86 (d, 1H, J=8.0 Hz), 7.38-7.50 (m, 3H,), 7.99 (d, 3H, J=7.1 Hz), 8.52 (s, 1H);

¹³C NMR (DMSO) δ 36.9, 62.3, 87.3, 87.9, 96.2, 109.4, 126.0, 129.1, 129.4, 131.0, 131.9 144.9. 145.1.

### EXAMPLE 4

### 2-(4″-NITROPHENYL)-6-(CIS-2′-HYDROXYMETHYL-5′-(1′,3′-OXATHIOLANYL))-IMIDAZO(1,2-C)PYRIMIDIN-5(6H)-ONE (compound #3)

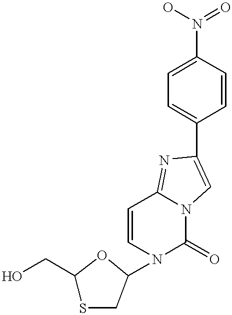

To a 50 ml flask equipped with a condenser were added 2-bromo-4′-nitroacetophenone (0.53 g, 2.16 mmol), (+/−)-2′-deoxy-3′-thiacytidine (0.311 g, 1.3 mmol), and methanol (30 ml). The reaction was refluxed for 48 hours . Upon cooling the reaction a yellow solid precipitated which was filtered and washed with methanol, methylene chloride and ethyl ether to yield 210 mg (41%) of the desired product as a yellow solid:

m.p. 232-234° C.;

¹H NMR (DMSO) δ 3.41 (d, 1H, J=4.2 Hz), 3.60 (dd, 1H, J=5.8, 12.1 Hz), 3.74 (d, 2H, J=4.4 Hz), 5.31 (t, 1H, J=4.4 Hz), 5.40 (bs, 1H), 6.53 (t, 1H, J=5.6 Hz), 6.81 (d, 1H, J=8.0 Hz), 7.85 (d, 1H, J=8.0 Hz), 8.30 (s, 4H), 8.71 (s, 1H);

¹³C NMR (DMSO) δ 36.5, 62.5, 86.9, 87.3, 98.1, 111.8, 124.1, 124.4, 126.7, 129.5, 129.8, 139.8, 142.1, 145.5, 145.8, 146.9.

### EXAMPLE 5

### 2-(4′-METHOXYPHENYL)-6-[CIS-2′-HYDROXYMETHYL-5′-(1′,3′-OXATHIOLANYL)]-IMIDAZO(1,2-C)PYRIMIDIN-5(6H)-ONE (compound #4)

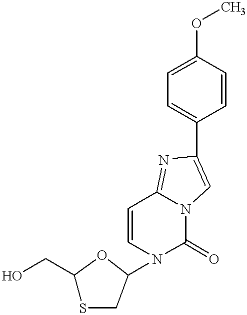

To a 50 ml flask equipped with a condenser were added α-bromo-p-methoxyacetophenone (1.42 g, 6.24 mmol), (+/−)-2′-deoxy-3′-thiacytidine (0.358 g, 1.56 mmol), and methanol (30 ml). The reaction was refluxed for 20 hours at which time water (15 ml) was added to the refluxing solution. After cooling the excess α-bromo-p-methoxy-acetophenone was removed by filtration and the filtrate was concentrated. The remaining aqueous layer was extracted with dichloromethane (3×), dried over MgSO₄ and adsorbed on silica gel. After purification by column chromatography (gradient eluting with 50% ethyl acetate/ hexanes to 100% ethyl acetate) 150 mg (26%) of light brown solid was isolated which was then washed with methanol to give 80 mg of the desired product as a white solid:

m.p. 194-196° C.;

¹H NMR (DMSO) δ 3.41 (d, 1H, J=4.2 Hz), 3.60 (dd, 1H, J=5.8, 12.1 Hz), 3.85 (m, 5H), 5.31 (t, 1H, J=4.4 Hz), 5.40 (5, 1H, J=5.0 Hz), 6.53 (t, 1H, J=5.6 Hz), 6.75 (d, 1H, J=8.0 Hz), 7.0 (d, 2H, J=7.2 Hz), 7.75 (d, 1H, J=8.0 Hz), 7.95 (d, 2H, J=7.2 Hz), 8.25 (s, 1H);

¹³C NMR (DMSO) δ 36.4, 55.3, 62.8, 86.9, 87.0, 98.3, 107.5, 114.5, 125.8, 127.3, 128.9, 144.4, 145.2, 145.6, 159.7.

### EXAMPLE 6

### 2-(2′-NAPHTHYL)-6-(CIS-2′-HYDROXYMETHYL-5′-(1′,3′-OXATHIOLANYL))-IMIDAZO(1,2-C)PYRIMIDIN-5(6H)-ONE (compound #5)

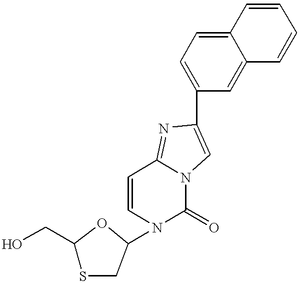

(+/−)-2′-Deoxy-3′-thiacytidine (200 mg, 0.873 mmol), 2-bromo-2′-acetonaphthone (261 mg, 1.048 mmol), and methanol (20 ml) were combined in a 50 ml flask equipped with a condenser and heated at reflux under nitrogen for 22 hours. After cooling the reaction mixture 136 mg (41 %) of the desired product was isolated by filtration as a white solid:

m.p. 224-226° C.;

¹H NMR (DMSO) δ 3.41 (d, 1H, J=4.2 Hz), 3.60 (dd, 1H, J=5.8, 12.1 Hz), 3.82 (d, 2H, J=4.4 Hz), 5.31 (t, 1H, J=4.4 Hz), 6.53 (t, 1H, J=5.6 Hz), 6.80 (d, 1H, J=8.2 Hz), 7.51-7.55 (m, 2H), 7.85 (d, 1H, J=8.0 Hz), 7.92-8.00 (m, 3H), 8.16 (d, 1H, J=8.6 Hz), 8.52 (s, 1H), 8.57 (s, 1H);

¹³C NMR (DMSO) δ 36.5, 62.7, 87.0, 87.2, 98.2, 109.5, 124.3, 124.5, 126.5, 126.8, 128.0, 128.5, 128.6, 129.5, 130.6, 133.1, 133.6, 144.2, 145.5, 145.6.

### EXAMPLE 7

### 2-METHYL-6-[CIS-2′-HYDROXYMETHYL-5′-(1′,3′-OXATHIOLANYL)]-IMIDAZO(1,2-C)PYRIMIDIN-5(6H)-ONE (compound #6)

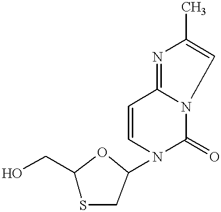

To a 25 ml flask equipped with a condenser were added chloroacetone (1.3 ml, 1.48 g, 16.0 mmol), (+/−)-2′-deoxy-3′-thiacytidine (0.458 g, 2.00 mmol), and methanol (10 ml). The reaction was refluxed for 19 hours. The reaction mixture was adsorbed directly on silica gel (2.0 g) and purified by column chromatography eluting with 5-10% methanol/dichloromethane to yield 98 mg of the desired product as a white solid:

m.p. 44-45° C.;

¹H NMR (CDCl₃) d 2.36 (s, 3H), 3.18 (dd, 1H, J=5.8, 11.6 Hz), 3.53 (dd, 1H, J=5.6, 11.7 Hz), 3.90-4.10 (m, 2H), 5.38 (t, 1H, J=3.1 Hz), 6.58 (t, 1H, J=5.5 Hz), 6.6 (d, 1H, J=8.0 Hz), 7.44 (s, 1H), 7.52 (d, 1H, J=8.0 Hz));

¹³C NMR (CDCl₃) d 13.9, 37.2, 63.6, 85.9, 87.2 99.0, 109.2, 126.8, 142.6, 144.3, 145.3;

UV (MeOH) 206 nm.

### EXAMPLE 8

### 2-(4″-FLUOROPHENYL)-6-CIS [2′-HYDROXYMETHYL-5′-(1′,3′-OXATHIOLANYL)]-IMIDAZO(1,2-C)PYRIMIDIN-5(6H)-ONE (compound #7)

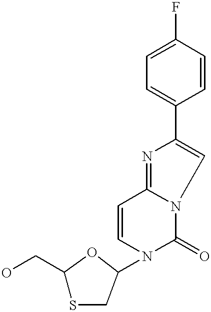

To a 25 ml flask equipped with a condenser were added 2-chloro-4′-fluoroacetophenone (0.696 g, 4.0 mmol), (+/−)-2′-deoxy-3′-thiacytidine (0.231 g, 1.0 mmol), and methanol (30 ml). The reaction was refluxed for 42 hours. The reaction mixture was then adsorbed directly on silica gel (1.0 g) and purified by column chromatography eluting with ethyl acetate/hexanes (4/1) to yield 118 mg (34%) of the desired product as a white solid:

m.p. 200=202° C.;

¹H NMR (DMSO) d 3.37 (dd, 1H, J=4.3, 12.8 Hz), 3.58 (dd, 1H, J=5.8, 12.0 Hz), 3.80 (t, 2H, J=5.3 Hz), 5.28 (t, 1H, J=4.5 Hz), 5.42 (t, 1H, J=5.8 Hz), 6.50 (dd, 1H, J=4.4, 5.8 Hz), 6.76 (d, 1H, J=8.0 Hz), 7.26 (t, 2H, J=9.0 Hz), 7.80 (d, 1H, J=7.8 Hz)); 8.00 (dd, 2H, J=6.6, 8.8 Hz), 8.38 (s, 1H);

¹³C NMR (DMSO) 36.4, 62.5, 86.7, 86.9, 98.0, 108.4, 115.6 (d, J=_{CF} 21.7 Hz), 127.5 (d, J=_{CF} 8 Hz), 128.8, 129.4, 143.0, 144.9, 145.1, 161.9 (d, J=_{CF} 244.7 Hz);

UV (MeOH) 277, 240. 205 nm.

### EXAMPLE 9

### 6-[2′-HYDROXYMETHYL-5′-(1′,3′-OXATHIOLANYL)]-IMIDAZO(1,2-C)PYRIMIDIN-5(6H)-ONE (compound #8)

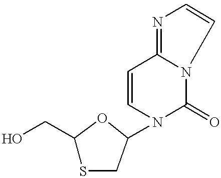

To a 100 ml flask equipped with a condenser were added chloroacetaldehyde (50% by wt. in water, 0.49 ml, 3.84 mmol), (+/−)-2′-deoxy-3′-thiacytidine (0.220 g, 0.96 mmol), and deionized water (20 ml). The reaction was refluxed for 10.5 hours. After concentrating the reaction mixture, the resulting residue was dissolved with methanol (50 ml) and adsorbed directly on silica gel. After purification by column chromagraphy (eluting with 5% methanol/dichloromethane), 33 mg (13.5%) of the desired product was isolated as a white solid:

m.p. 162-163° C.;

¹H NMR (DMSO) d 3.33 (dd, 1H, J=4.2, 12.0 Hz), 3.56 (dd, 1H, J=5.8, 12.0 Hz), 3.78 (t, 2H, J=4.9 Hz), 5.27 (t, 1H, J=4.5 Hz), 5.39 (t, 1H, J=7.7 Hz), 6.47 (dd, 1H, J=4.4, 5.8 Hz), 6.73 (d, 1H, J=8.0 Hz), 7.40 (d, 1H, J=1.5 Hz), 7.76 (d, 1H, J=8.0 Hz), 7.82 (bd, 1H, J=1.2 Hz);

¹³C NMR (DMSO) d 36.2, 62.6, 86.6, 86.7, 98.3, 112.8, 128.1, 132.6, 144.5, 145.3;

UV (MeOH) 271.1, 213 nm.

### EXAMPLE 10

### ETHYL-6-(TRANS-2′-TERTBUTYLDIMETHYLSILYLOXYMETHYL-5′-(1′,3′-OXATHIOLANYL))-IMIDAZO(1,2-C)PYRIMIDIN-5(6H)-ONE-2-CARBOXYLATE

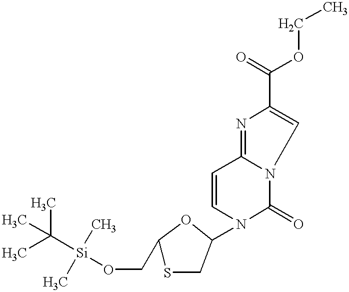

A solution of ethyl-6-(trans-2′-hydroxymethyl-5′-(1′,3′-oxathiolanyl))-imidazo(1,2-c)pyrimidin-5(6H)-one-2-carboxylate (500 mg, 1.69 mmol), imidazole (230 mg, 3.38 mmol), tert-butyldimethylsilyl chloride (380 mg, 2.53 mmol), and DMF (7 ml) was stirred at room temperature for 22 hours. After concentration of the the reaction mixture, the remaining residue was dissolved in methanol, adsorbed on silica gel and purified by column chromatography to yield 488 mg (73%) of the desired product as a clear, coloroless oil:

¹H NMR (CDCl₃) d 0.11 (s, 6H), 0.93 (s, 9H), 1.42 (t, 3H, J=7.1 Hz), 3.18 (dd, 1H, J=2.42, 12.1 Hz), 3.70 (dd, 1H, J=5.7, 12.1 Hz), 3.76 (m, 2H, ), 4.44 (q, 2H, J=14.3, 7.1 Hz), 5.59 (t, 1H, J=4.18 Hz), 6.68 (d, 1H, J=8.0 Hz), 6.72 (dd, 1H, J=2.4, 5.7 Hz), 7.39 (d, 1H, J=8.0 Hz), 8.33 (s, 1H).

### EXAMPLE 11

### 2-HYDROXYMETHYL-6-(TRANS-2′-TERT-BUTYLDIMETHYLSILYLOXYMETHYL-5-(1′,3′-OXATHIOLANYL))-IMIDAZO(1,2-C)PYRIMIDIN-5(6H)-ONE

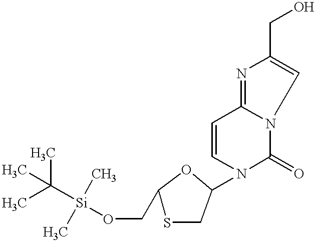

To a slurry of lithium aluminum hydride (19 mg, 0.52 mmol) in tetrahydrofuran (30 ml) at 0° C. under nitrogen atmosphere, was added a solution of ethyl-6-(trans-2′-tert-butyldimethylsilyloxymethyl-5′-(1′,3′-oxathiolanyl))-imidazo(1,2-c)pyrimidin-5(6H)-one-2-carboxylate (410 mg, 1.03 mmol) in tetrahydrofuran (10 ml). The reaction was stirred at room temperature for 2.5 hours at which time 1.5 ml of saturated ammonium chloride solution was added. The reaction was concentrated and the remaining residue was dissolved in dichloromethane, adsorbed on silica gel and purified by column chromatography to yield 191 mg (53%) of the desired product as an oil:

¹H NMR (CDCl₃) δ 0.10 (s, 3H), 0.11 (s, 3H), 0.92 (s, 9H), 3.16 (dd, 1H, J=2.6, 12.1 Hz), 3.67 (dd, 1H, J=5.7, 12.5 Hz), 3.74-4.13 (m, 2H), 4.77 (s.2H), 5.58 (t, 1H, J=4.3 Hz), 6.60 (d, 1H, J=8.0 Hz), 6.73 (dd, 1H, J=2.6, 5.8 Hz), 7.35 (d, 1H, J=8.0 Hz), 7.61 (s, 1H).

### EXAMPLE 12

### 2-HYDROXYMETHYL-6-(TRANS-2′-HYDROXYMETHYL-5′-(1′,3′-OXATHIOLANYL))-IMIDAZO(1,2-C)PYRIMIDIN-5(6H)-ONE (compound #9)

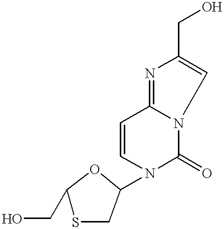

A solution of 2-hydroxymethyl-6-(trans-2′-tert-butyldimethylsilyloxymethyl-5′-(1′,3′-oxathiolanyl))-imidazo(1,2-c)pyrimidin-5(6H)-one (191 mg, 0.54 mmol), tetrabutylammonium flluoride (1.0 molar, 0.81 ml, 0.8 mmol), acetic acid (60 μl, 1.0 mmol) and tetrahydrofuran (6 ml) was stirred for 18 hours at room temperature. The reaction mixture was adsorbed on silica gel and purified by column chromatography to yield 70 mg (45%) of the desired product as a white solid:

m.p. 163-164° C.;

¹H NMR (DMSO) d 3.36 (dd, 1H, J=2.8 12.3 Hz), 3.53-3.65 (m, 3H), 3.67 (d, 2H, J=4.9 Hz), 5.19-5.25 (m, 2H). 5.61 (t, 1H, J=5.2 Hz), 6.60-6.65 (m, 2H), 7.51 (d, 1H, J=8.0 Hz), 7.58 (s, 1H);

¹³C NMR (DMSO) d 36.1, 67.7, 64.7, 87.4, 88.0, 97.8, 108.7, 128.3, 144.3, 145.1, 147.3.

### EXAMPLE 13

### ETHYL-6-[2′R-HYDROXYMETHYL-5′S-(1′,3′-OXATHIOLANYL)]-IMIDAZO(1,2-C)PYRIMIDIN-5(6H)-ONE-2-CARBOXYLATE (compound #10)

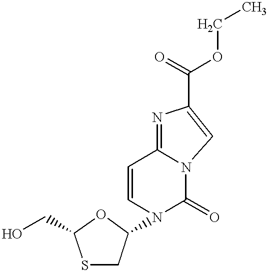

To a 100 ml flask equipped with a condenser were added ethyl bromopyruvate (1.92 ml, 2.99 g, 15.33 mmol), (−)-2′-deoxy-3′-thiacytidine(2R,5S) (0.878 g, 3.83 mmol), and methanol (80 ml). The reaction was refluxed for 20 hours. After removing the methanol, the remaining residue was dissolved with dichloromethane (50 ml) and adsorbed directly on silica gel. After purification by column chromagraphy (eluting with ethyl acetate) 262 mg (21 %) of the the ethyl ester with ca. 10% contamination by the methyl ester was isolated as a yellow solid:

m.p. 141-143° C.;

¹H NMR (CDCl₃) δ 1.41 (t, 3H, J=7.1 Hz), 3.18 (dd, 1H, J=5.5, 11.8 Hz), 3.53 (dd, 1H, J=5.6, 12.3 Hz), 3.94 (dd, H, J=3.8, 12.6 Hz), 4.10 (dd, 1H, J=2.5, 12.0 Hz), 4.42 (q, 2H, J=7.2 Hz), 5.36 (t, 1H, J=2.7 Hz), 6.54 (t, 1H, J=5.5 Hz), 6.61 (d, 1H, J=8.0 Hz), 7.66 (d, 1H, J=8.0 Hz), 8.31 (s, 1H);

¹³C NMR (CDCl₃) δ 14.9, 37.3, 61.2, 66.7, 86.5, 87.3, 99.5, 118.0, 129.0, 136.8, 145.3, 145.5, 162.4.

### EXAMPLE 14

### ETHYL-6-[-2′R-TERT-BUTYLDIMETHYLSILYLOXYMETHYL-5′S-(1′,3′-OXATHIOLANYL)]-IMIDAZO(1,2-C)PYRIMIDIN-5(6H)-ONE-2-CARBOXYLATE

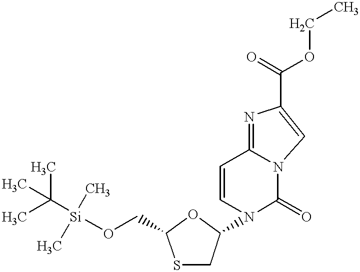

A solution of ethyl-6-(2′R-hydroxymethyl-5′S-(1′,3′-oxathiolanyl))-imidazo(1,2-c)pyrimidin-5(6H)-one-2-carboxylate (example 7) (148 mg, 0.502 mmol), triethylamine (0.14 ml, 102 mg, 1.00 mmol), tert-butyldimethylsilyl chloride (90 mg, 0.602 mmol), and dichloromethane (10 ml) was stirred at room temperature for 22 hours. After addition of methanol (0.5 ml) the reaction mixture was adsorbed on silica gel and purified by column chromatography to yield 90 mg (45%) of the desired product as a clear, colorless oil:

¹H NMR (CDCl₃) δ 0.12 (s, 6H), 0.93 (s, 9H), 1.41 (t, 3H, J=7.1 Hz), 3.19 (dd, 1H, J=4.2, 12.2 Hz), 3.53 (dd, 1H, J=5.5, 12.2 Hz), 3.95 (dd, 1H, J=3.3, 11.6 Hz), 4.09-4.15 (m, 1H), 4.44 (q, 2H, J=14.3, 7.1 Hz), 5.30 (t, 1H, J=3.3 Hz), 6.53 (t, 1H, J=4.4 Hz), 6.64 (d, 1H, J=8.0 Hz), 7.88 (d, 1H, J=8.1 Hz), 8.31 (s, 1H).

### EXAMPLE 15

### 2-HYDROXYMETHYL-6-[-2′R-TERT-BUTYLDIMETHYLSILYLOXYMETHYL-5′S-(1′,3′-OXATHIOLANYL)]-IMIDAZO(1,2-C)PYRIMIDIN-5(6H)-ONE

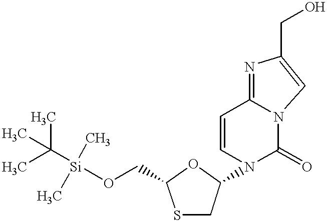

A slurry of lithium aluminum hydride (10 mg, 0.226 mmol) in tetrahydrofuran (10 ml) at 0° C. under nitrogen was added a solution of ethyl-6-[2′R-tert-butyidimethylsilyloxymethyl-5′S-(1′,3′-oxathiolanyl)]-imidazo(1,2-C)pyrimidin-5(6H)-one-2-carboxylate (example 8) (90 mg, 0.226 mmol) in tetrahydrofuran (15 ml). The reaction was stirred at room temperature for 2.5 hours at which time 0.5 mg of saturated ammonium chloride solution was added. The reaction was concentrated and the remaining residue was dissolved in dichloromethane, adsorbed on silica gel and purified by column chromatography to yield 34 mg (43%) of the desired product as an oil:

¹H NMR (CDCl₃) δ 0.137 (s, 6H), 0.94 (s, 9H), 3.16 (dd, 1H, J=4.6, 12.0 Hz), 3.58 (dd, 1H, J=5.5, 12.1 Hz), 3.95 (dd, 1H, J=3.5, 11.5 Hz), 4.10 (dd, 1H, J=3.5, 11.5 Hz), 4.70 (s, 2H), 5.30 (t, 1H, J=3.6 Hz), 6.56 (t, 1H, J=4.6 Hz), 6.57 (d, 1H, J=8.0 Hz), 7.65 (s, 1H), 7.79 (d, 1H, J=8.0 Hz).

### EXAMPLE 16

### (−)-2-HYDROXYMETHYL-6-(-2′R-HYDROXYMETHYL-5′S-(1′,3′-OXATHIOLANYL))-IMIDAZO(1,2-C)PYRIMIDIN-5(6H)-ONE (compound #11)

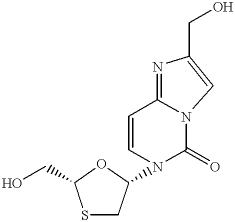

A solution of 2-hydroxymethyl-6-(2′R-tert-butydimethylsilyloxymethyl-5′S-(1′,3′-oxathiolanyl))-imidazo(1,2-c)pyrimidin-5(6H)-one (example 9) (34 mg, 0.10 mmol), tetrabutylammonium fluoride (1.0 molar, 0.153 ml, 0.153 mmol), acetic acid (12 μl, 0.2 mmol) and tetrahydrofuran (10 ml) was stirred for 18 hours at room temperature. The reaction mixture was adsorbed on silica gel and purified by column chromatography to yield 21 mg of oil which was lyophilized to yield 21 mg (74%) of the desired product as a white solid:

[α]_{D}²³−63 (c=0.1, MeOH);

m.p. 64-65° C.;

¹H NMR (CD₃OD) δ 3.08 (dd, 1H, J=4.6, 11.8 Hz), 3.37 (dd, 1H, J=5.6, 12.0 Hz), 3.67 (dd, 1H, J=4.4, 12.4 Hz), 3.75 (dd, 1H, J=3.7, 12.4 Hz) 4.43 (s, 2H), 5.12 (t, 1H, J=3.7 Hz), 6.34 (t, 1H, J=5.5 Hz), 6.39 (d, 1H, J=8.0 Hz), 7.45 (s, 1H), 7.70 (d, 1H, J=8.0 Hz);

¹³C NMR (CD₃OD) δ 37.8, 59.0, 64.3, 87.9, 88.7, 98.6, 110.7, 130.4, 146.7, 146.9, 147.3.

### EXAMPLE 17

### 2-PHENYL-6-(2′R-HYDROXYMETHYL-5′S-(1′,3′-OXATHIOLANYL)-IMIDAZO(1,2-C)PYRIMIDIN-5(6H)-ONE (compound #12)

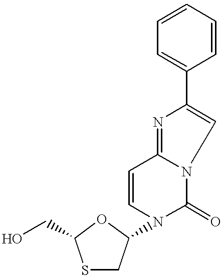

To a 25 ml flask equipped with a condenser were added 2-bromoacetophenone (0.478 g, 2.4 mmol), (−)-2′-deoxy-3′-thiacytidine (0.250 g, 1.1 mmol), and methanol (20 ml). The reaction was refluxed for 20 hours. The reaction mixture was adsorbed directly on silica gel (1.0 g) and purified by column chromatography eluting with ethyl acetate/hexanes (4/1) to yield 91 mg (26%) of the desired product as a white solid:

[α]_{D}=−76 (c=0.15, t=23° C., HOAc);

m.p. 182-184° C.;

¹H NMR (DMSO) d 3.40 (dd, 1H, J=3.6, 12.1 Hz), 3.62 (dd, 1H, J=5.8, 12.2 Hz), 3.80 (t, 2H, J=4.1 Hz), 5.28 (t, 1H, J=4.4 Hz), 5.42 (t, 1H, J=5.7 Hz), 6.50 (dd, 1H, J=5.4, 5.8 Hz), 6.80 (d, 1H, J=8.0 Hz), 7.31-7.59 (m, 3H), 7.89 (d, 1H, J=8.0 Hz); 8.00 (d, 2H, J=7.0 Hz), 8.40 (s, 1H);

UV (MeOH) 286, 240, 108 nm.

### EXAMPLE 18

### 2-(4″-NITROPHENYL-6-(2′R-HYDROXYMETHYL-5′S-(1′,3′-OXATHIOLANYL)-IMIDAZO(1,2-C)PYRIMIDIN-5(6H)-ONE (compound #13)

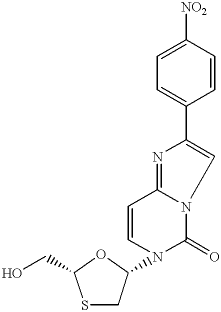

To a 25 ml flask equipped with a condenser were added 2-bromo-4′-nitroacetophenone (0.426 g, 1.75 mmol), (−)-2′-deoxy-3′-thiacytidine (0.250 g, 1.1 mmol), and methanol (20 ml). The reaction was refluxed for 20 hours. After cooling the reaction 118 mg (28%) of the desired product was isolated by filtration as a yellow solid:

[α]_{D}=−68 (c=0.21, t=21° C., HOAc);

m.p. 234-236° C.;

¹H NMR (DMSO) d 3.41 (d, 1H, J=4.2 Hz), 3.60 (dd, 1H, J=5.8, 12.1 Hz), 3.74 (d, 2H, J=4.4 Hz), 5.31 (t, 1H, J=4.4 Hz), 5.40 (bs, 1H), 6.53 (t, 1H, J=5.6 Hz), 6.81 (d, 1H, J=8.0 Hz), 7.85 (d, 1H, J=8.0 Hz), 8.30 (s, 4H), 8.71 (s, 1H);

UV (MeOH) 335, 274, 226, 207 nm.

### EXAMPLE 19

### 2-(4″-FLUOROPHENYL-6-(2′R-HYDROXYMETHYL-5′S-(1′,3′-OXATHIOLANYL)-IMIDAZO(1,2-C)PYRIMIDIN-5(6H)-ONE (compound #14)

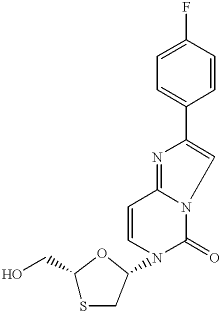

To a 25 ml flask equipped with a condenser were added 2-chloro-4′-fluoroacetophenone (0.696 g, 4.0 mmol), (−)-2′-deoxy-3′-thiacytidine (0.231 g, 1.0 mmol), and methanol (30 ml). The reaction was refluxed for 48 hours. The reaction mixture was adsorbed directly on silica gel (1.0 g) and purified by column chromatography eluting with ethyl acetate/hexanes (4/1) to yield 91 mg (26%) of the desired product as a white solid:

[α]=−67 (c=0.11, t=23° c., HOAc);

m.p. 182-184° C.;

¹H NMR (DMSO) d 3.37 (dd, 1H, J=4.3, 12.8 Hz), 3.58 (dd, 1H, J=5.8, 12.0 Hz), 3.80 (t, 2H, J=5.3 Hz), 5.28 (t, 1H, J=4.5 Hz), 5.42 (t, 1H, J=5.8 Hz), 6.50 (dd, 1H, J=4.4, 5.8 Hz), 6.76 (d, 1H, J=8.0 Hz), 7.26 (t, 2H, J=9.0 Hz), 7.80 (d, 1H, J=7.8 Hz)); 8.00 (dd, 2H, J=6.6, 8.8 Hz), 8.38 (s, 1H); UV (MeOH) 277, 240. 205 nm.

### EXAMPLE 20

### ETHYL-6-(-2′S-TERTBUTYLDIMETHYLSILYLOXYMETHYL-5′R-(1′,3′-OXATHIOLANYL))-IMIDAZO(1,2-C)PYRIMIDIN-5(6H)-ONE-2-CARBOXYLATE

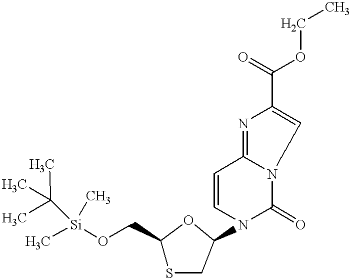

A solution of ethyl-6-(2′S-hydroxymethyl-5′R-(1′,3′-oxathiolanyl ))-imidazo(1,2-c)pyrimidin-5(6H)-one-2-carboxylate (148 mg, 0.502 mmol), imidazole (92 mg, 11.36 mmol), tert-butyidimethylsilyl chloride (152 mg, 1.017 mmol), and DMF (5 ml) was stirred at room temperature for 22 hours. After addition of methanol (0.5 ml) the reaction mixture was concentrated under high vacuum, adsorbed on silica gel and purified by column chromatography to yield 141 mg (52%) of the desired product as a clear, coloroless oil:

¹H NMR (CDCl₃) d 0.12 (s, 6H), 0.93 (s, 9H), 1.41 (t, 3H, J=7.1 Hz), 3.19 (dd, 1H, J=4.2, 12.2 Hz), 3.53 (dd, 1H, J=5.5, 12.2 Hz), 3.95 (dd, 1H, J=3.3, 11.6 Hz), 4.09-4.15 (m, 1H), 4.44 (q, 2H, J=14.3, 7.1 Hz), 5.30 (t, 1H, J=3.3 Hz), 6.53 (t, 1H, J=4.4 Hz), 6.64 (d, 1H, J=8.0 Hz), 7.88 (d, 1H, J=8.1 Hz), 8.31 (s, 1H).

### EXAMPLE 21

### 2-HYDROXYMETHYL-6-(-2′S-TERT-BUTYLDIMETHYLSILYLOXYMETHYL-5′R-(1′,3′-OXATHIOLANYL))-IMIDAZO(1,2-C)PYRIMIDIN-5(6H)-ONE

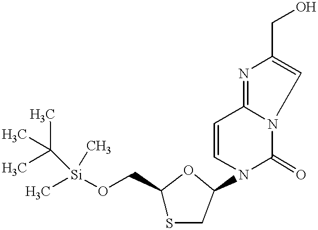

To a slurry of lithium aluminum hydride (10 mg, 0.226 mmol) in tetrahydrofuran (10 ml) at 0° C. under nitrogen atmosphere, was added a solution of ethyl-6-(2′S-tert-butyldimethylsilyloxymethyl-5′R-(1′,3′-oxathiolanyl))-imidazo(1,2-c)pyrimidin-5(6H)-one-2-carboxylate (141 mg, 0.355 mmol) in tetrahydrofuran (20 ml). The reaction was stirred at room temperature for 2.5 hours at which time 0.5 ml of saturated ammonium chloride solution was added. The reaction was concentrated and the remaining residue was dissolved in dichloromethane, adsorbed on silica gel and purified by column chromatography to yield 124 mg (99%) of the desired product as an oil:

¹H NMR (CDCl₃) δ 0.14 (s, 6H), 0.94 (s, 9H), 3.16 (dd, l H, J=4.6, 12.0 Hz), 3.58 (dd, 1H, J=5.5, 12.1 Hz), 3.95 (dd, 1H, J=3.5, 11.5 Hz), 4.10 (dd, 1H, J=3.5, 11.5 Hz), 4.70 (s, 2H), 5.30 (t, 1H, J=3.6 Hz), 6.56 (t, 1H, J=4.6 Hz), 6.57 (d, 1H, J=8.0 Hz), 7.65 (s, 1H), 7.79 (d, 1H, J=8.0 Hz).

### EXAMPLE 22

### (−)-2-HYDROXYMETHYL-6-(-2′S-HYDROXYMETHYL-5′R-(1′,3′-OXATHIOLANYL))-IMIDAZO(1,2-C)PYRIMIDIN-5(6H)-ONE (compound #15)

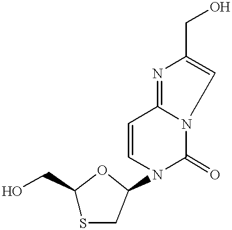

A solution of 2-hydroxymethyl-6-(2′S-tert-butyldimethylsilyloxymethyl-5′R-(1′,3′-oxathiolanyl))-imidazo(1,2-c)pyrimidin-5(6H)-one (124 mg, 0.35 mmol), tetrabutylammonium flluoride (1.0 molar, 0.53 ml, 0.526 mmol), acetic acid (40 μl, 0.7 mmol) and tetrahydrofuran (8 ml) was stirred for 18 hours at room temperature. The reaction mixture was adsorbed on silica gel and purified by column chromatography to yield 28 mg of an oil which was further purified by HPLC abd lyopholized to yield 20 (20%) of the desired product as a white solid:

[α]_{D}²³+60 (c=0.1, MeOH);

m.p. 64-65° C.; ¹H NMR (CD₃OD) d 3.08 (dd, 1H, J=4.6, 11.8 Hz), 3.37 (dd, 1H, J=5.6, 12.0 Hz), 3.67 (dd, 1H, J=4.4, 12.4 Hz), 3.75 (dd, 1H, J=3.7, 12.4 Hz) 4.43 (s, 2H), 5.12 (t, 1H, J=3.7 Hz), 6.34 (t, 1H, J=5.5 Hz), 6.39 (d, 1H, J=8.0 Hz), 7.45 (s, 1H), 7.70 (d, 1H, J=8.0 Hz);

¹³C NMR (DMSO) d 37.8, 59.0, 64.3, 87.9, 88.7, 98.6, 110.7, 130.4, 146.7, 146.9, 147.3.

Other compounds have been synthesized accordingly:

TABLE I
The following cis compound were produced as racemic mixtures.

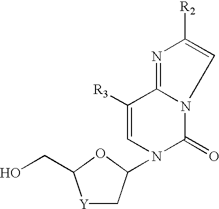

| Compound # | R₂ | Y | R₃ |
|---|---|---|---|
| 16 | —COOEt | S | H |
| 17 | 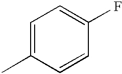 | O | H |
| 18 | —CH₂OH | O | H |
| 19 | —COOMe | O | H |
| 20 | —CH₂OH | O | F |
| 21 | 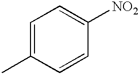 | O | H |
| 22 | 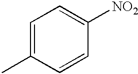 | O | F |

TABLE 2
The following trans compounds were produced as racemic mixtures.

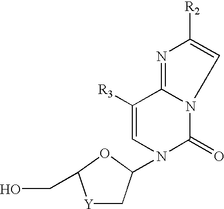

| Compound # | R₂₂ | Y | R₃ |
|---|---|---|---|
| 23 | —CH₂OH | O | F |
| 24 | —CH₂Me | O | H |
| 25 | —CH₂OH | O | H |
| 26 | NO₂ | O | H |
| 27 | NO₂ | O | F |

TABLE 3
Compound #28 was produced as a pure enantiomer.

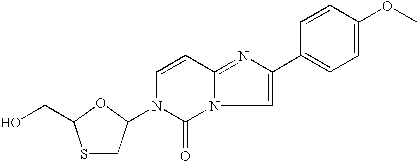

| Compound # | R₂ | Y | R₃ |
|---|---|---|---|
| 28 | 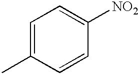 | —CH₂ | H |

The present invention also includes the following nucleosides:

| | | |
|---|---|---|
| 29 | 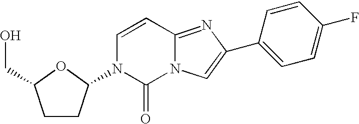 | 6-[(2S,5R)-5-HYDROXYMETHYL OXOLAN-2-YL]-2-(4-FLUORO PHENYL)-5,6-DIHYDRO[1,3]- DIAZOLO[1,2-C][1,3]DIAZIN-5-ONE |
| 30 | 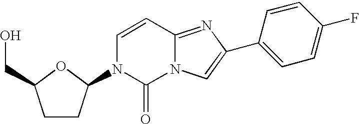 | 6-[(2R,5S)-5-HYDROXYMETHYL OXOLAN-2-YL]-2-(4-NITROPHENYL)- 5,6-DIHYDRO[1,3]-DIAZOLO[1,2- C][1,3]DIAZIN-5-ONE |
| 31 | 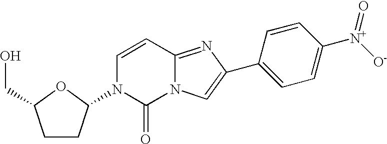 | 6-[(2S,5R)-5-HYDROXYMETHYL OXOLAN-2-YL]-2-(4-NITROPHENYL)- 5,6-DIHYDRO[1,3]-DIAZOLO[1,2- C][1,3]DIAZIN-5-ONE |
| 32 | 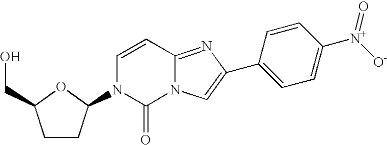 | 2-(4-NITROPHENYL)-6-[(2R,5S)-5- HYDROXYMETHYLOXOLAN-2-YL]- 5,6-DIHYDRO[1,3]-DIAZOLO[1,2- C][1,3]DIAZIN-5-ONE |
| 33 | 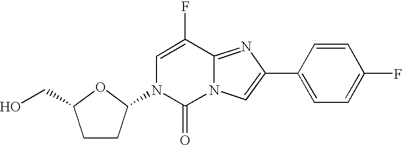 | 8-FLUORO-2-(4-FLUOROPHENYL)-6- [(5R,2S)-5-HYDROXYMETHYL OXOLAN-2-YL]-5,6-DIHYDRO [1,3]DIAZOLO[1,2-C][1,3]DIAZIN-5- ONE |
| 34 | 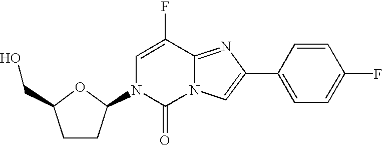 | 8-FLUORO-2-(4-FLUOROPHENYL)-6- [(2R,5S)-5-HYDROXYMETHYL OXOLAN-2-YL]-5,6-DIHYDRO[1,3]- DIAZOLO[1,2-C][1,3]DIAZIN-5-ONE |
| 35 | 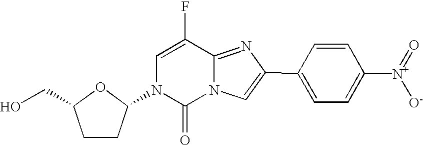 | 8-FLUORO-2-(4-NITROPHENYL)-6- [(5R,2S)-5-HYDROXYMETHYL OXOLAN-2-YL]-5,6-DIHYDRO [1,3]DIAZOLO[1,2-C][1,3]DIAZIN-5- ONE |
| 36 | 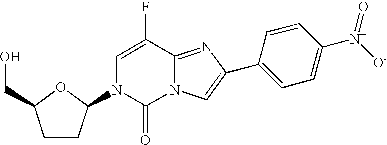 | 8-FLUORO-2-(4-NITROPHENYL)-6- [(2R,5S)-5-HYDROXYMETHYL OXOLAN-2-YL]-5,6-DIHYDRO[1,3]- DIAZOLO[1,2-C][1,3]DIAZIN-5-ONE |
| 37 | 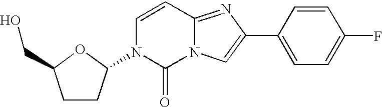 | 2-(4-FLUOROPHENYL)-6-[(2S,5S)-5- HYDROXYMETHYLOXOLAN-2-YL]- 5,6-DIHYDRO[1,3]-DIAZOLO[1,2- C][1,3]DIAZIN-5-ONE |
| 38 | 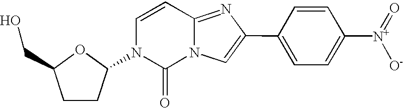 | 2-(4- NITROPHENYL)-6-[(2S,5S)-5- HYDROXYMETHYLOXOLAN-2-YL]- 5,6-DIHYDRO[1,3]-DIAZOLO[1,2- C][1,3]DIAZIN-5-ONE |
| 39 | 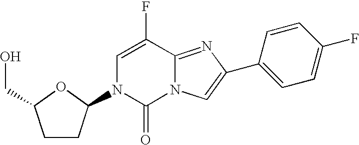 | 6-[(2R,5R)-5-HYDROXYMETHYL OXOLAN-2-YL]-2-(4-FLUORO PHENYL)-5,6-DIHYDRO[1,3]- DIAZOLO[1,2-C][1,3]DIAZIN-5-ONE |
| 40 | 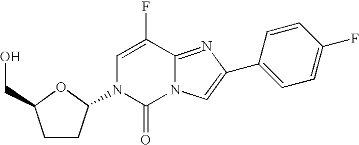 | 8-FLUORO-2-(4-FLUOROPHENYL)-6- [(2S,5S)-5-HYDROXYMETHYL OXOLAN-2-YL]-5,6-DIHYDRO[1,3]- DIAZOLO[1,2-C][1,3]DIAZIN-5-ONE |
| 41 | 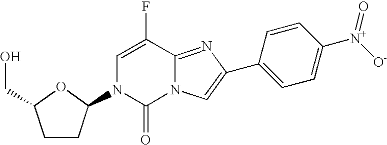 | 8-FLUORO-2-(4-NITROPHENYL)- [(2R,5R)-5-HYDROXYMETHYL OXOLAN-2-YL]-5,6-DIHYDRO[1,3]- DIAZOLO[1,2-C][1,3]DIAZIN-5-ONE |
| 42 | 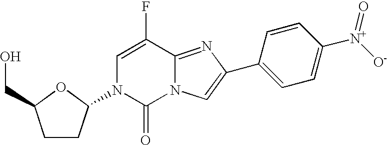 | 8-FLUORO-2-(4-NITROPHENYL)-6- [(2S,5S)-5-HYDROXYMETHYL OXOLAN-2-YL]-5,6-DIHYDRO[1,3]- DIAZOLO[1,2-C][1,3]DIAZIN-5-ONE |
| 43 | 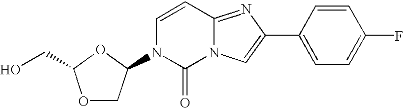 | 2-(4-FLUOROPHENYL)-6-[(2S,4S)-2- HYDROXYMETHYL-1,3-DIOXOLAN-4- YL]-5,6-DIHYDRO[1,3]DIAZOLO[1,2- C][1,3]DIAZIN-5-ONE |
| 44 | 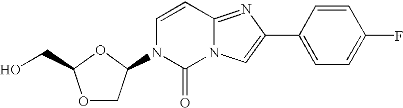 | 2-(4-FLUOROPHENYL)-6-[(2R,4R)-2- HYDROXYMETHYL-1,3-DIOXOLAN-4- YL]-5,6-DIHYDRO[1,3]DIAZOLO[1,2- C][1,3]DIAZIN-5-ONE |
| 45 | 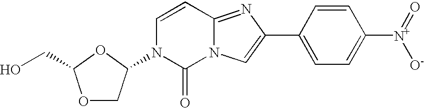 | L2-(4-NITROPHENYL)-6-[(2S,4S)-2- HYDROXYMETHYL-1,3-DIOXOLAN-4- YL]-5,6-DIHYDRO[1,3]DIAZOLO[1,2- C][1,3]DIAZIN-5-ONE |
| 46 | 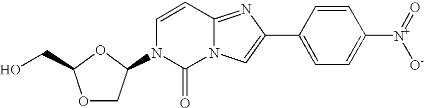 | 2-(4-NITROPHENYL)-6-[(2R,4R)-2- HYDROXYMETHYL-1,3-DIOXOLAN-4- YL]-5,6-DIHYDRO[1,3]DIAZOLO[1,2- C][1,3]DIAZIN-5-ONE |
| 47 | 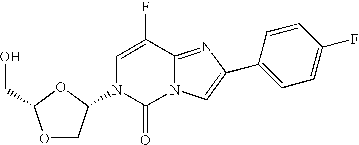 | 8-FLUORO-2-(4-FLUOROPHENYL)-6- [(2S,4S)-5-HYDROXYMETHYL OXOLAN-4-YL]-5,6-DIHYDRO[1,3]- DIAZOLO[1,2-C][1,3]DIAZIN-5-ONE |
| 48 | 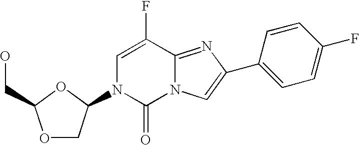 | 8-FLUORO-2-(4- FLUOROPHENYL)- [(2R,4R)-2-HYDROXYMETHYL-1,3- DIOXOLAN-4-YL]-5,6-DIHYDRO[1,3]- DIAZOLO[1,2-C][1,3]DIAZIN-5-ONE 8-FLUORO-2-(4-NITROPHENYL)-6- [(2S,4S)-5-HYDROXYMETHYL |
| 49 | 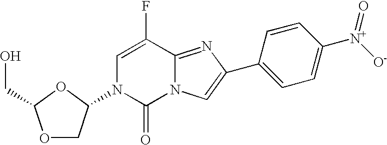 | OXOLAN-4-YL]-5,6-DIHYDRO[1,3]- DIAZOLO[1,2-C][1,3]DIAZIN-5-ONE |
| 50 | 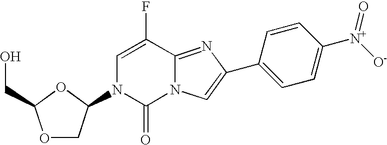 | 8-FLUORO-2-(4-NITROPHENYL)- [(2R,4R)-2-HYDROXYMETHYL-1,3- DIOXOLAN-4-YL]-5,6-DIHYDRO[1,3]- DIAZOLO[1,2-C][1,3]DIAZIN-5-ONE |
| 51 | 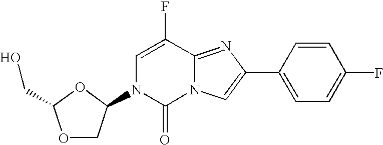 | 2-(4-FLUOROPHENYL)-6-[(2S,4R)-5- HYDROXYMETHYL-1,3-DIOXOLAN-4- YL]-5,6-DIHYDRO[1,3]-DIAZOLO[1,2- C][1,3]DIAZIN-5-ONE |
| 52 | 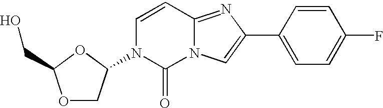 | 2-(4-FLUOROPHENYL)-6-[(2R,4S)-5- HYDROXYMETHYL-1,3-DIOXOLAN-4- YL]-5,6-DIHYDRO[1,3]-DIAZOLO[1,2- C][1,3]DIAZIN-5-ONE |
| 53 | 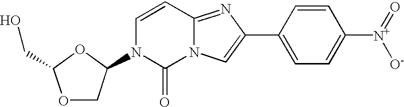 | 2-(4-NITROPHENYL)-6-[(2S,4R)-5- HYDROXYMETHYL-1,3-DIOXOLAN-4- YL]-5,6-DIHYDRO[1,3]-DIAZOLO[1,2- C][1,3]DIAZIN-5-ONE |
| 54 | 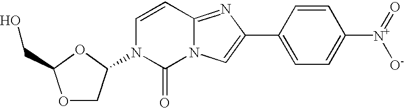 | 2-(4-NITROPHENYL)-6-[(2R,4S)-5- HYDROXYMETHYL-1,3-DIOXOLAN-4- YL]-5,6-DIHYDRO[1,3]-DIAZOLO[1,2- C][1,3]DIAZIN-5-ONE |
| 55 | 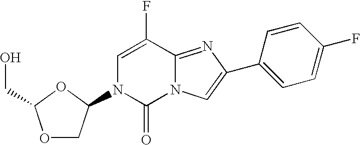 | 8-FLUORO-2-(4-FLUOROPHENYL)-6- [(2S,4R)-2-HYDROXYMETHYL-1,3- DIOXOLAN-4-YL]-5,6- DIHYDRO[1,3]DIOXOLO[1,2- C][1,3]DIAZIN-5-ONE |
| 56 | 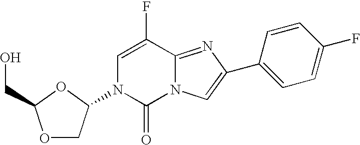 | 8-FLUORO-2-(4-FLUOROPHENYL)- [(2R,4S)-5-HYDROXYMETHYL OXOLAN-4-YL]-5,6-DIHYDRO[1,3]- DIAZOLO[1,2-C][1,3]DIAZIN-5-ONE |
| 57 | 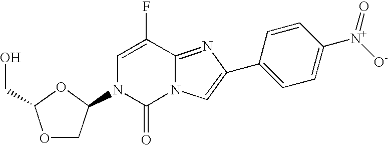 | 8-FLUORO-2-(4-NITROPHENYL)-6- [(2S,4R)-2-HYDROXYMETHYL-1,3- DIOXOLAN-4-YL]-5,6-DIHYDRO [1,3]DIAZOLO[1,2-C][1,3]DIAZIN-5- ONE |
| 58 | 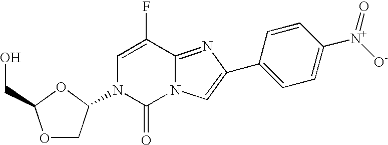 | 8-FLUORO-2-(4-NITROPHENYL)- [(2R,4S)-5-HYDROXYMETHYL OXOLAN-4-YL]-5,6-DIHYDRO[1,3]- DIAZOLO[1,2-C][1,3]DIAZIN-5-ONE |
| 59 | 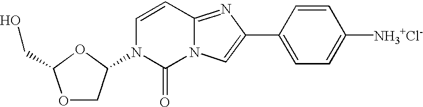 | 4-(6-(2S,5R)-5-HYDROXYMETHYL OXOLAN-2-YL-(5-OXO-5,6- DIHYDRO[1,3-DIAZOLO[1,2-C][1,3]- DIAZIN-2-YL)PHENYLAMMONIUM CHLORIDE |
| 60 | [chemical structure] | 2-AMINO-3-METHYL-BUTYRIC ACID 4-[2-(4-FLUORO-PHENYL)-5-OXO- IMIDAZO[1,2-C]PYRIMIDIN-6-YL]- [1,3]DIOXOLAN-2-YLMETHYL ESTER; HYDROCHLORIDE SALT |
| 61 | 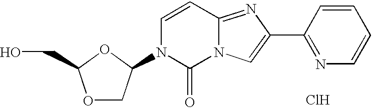 | 6-(2-HYDROXYMETHYL-[1,3]DIOXOLAN-4-YL)-2-PYRIDIN-2-YL-6H- IMIDAZO[1,2-C]PYRIMIDIN-5-ONE; HYDROCHLORIDE SALT |
| 62 | 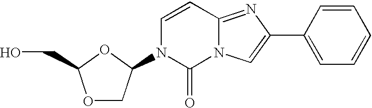 | 6-(2-HYDROXYMETHYL- [1,3]DIOXOLAN-4-YL)-2-PHENYL-6H- IMIDAZO[1,2-C]PYRIMIDIN-5-ONE 6-(2-HYDROXYMETHYL- |
| 63 | 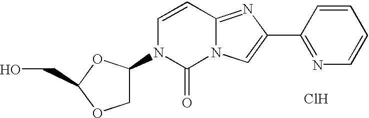 | [1,3]DIOXOLAN-4-YL)-2-PYRIDIN-2- YL-6H-IMIDAZO[1,2-C]PYRIMIDIN-5- ONE |
| 64 | 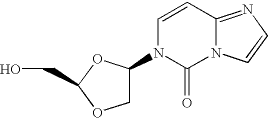 | 6-(2-HYDROXYMETHYL-[1,3]DIOXOLAN-4-YL)-6H-IMIDAZO[1,2- C]PYRIMIDIN-5-ONE |
| 65 | [chemical structure] | 6-(2-HYDROXYMETHYL-(1,3,) DIOXOLAN-4-YL)-3-PYRIDIN-3-YL-6H- IMIDAZO(1,2)PRIMIDIN-5-ONE |

HIV-1 infection assays with cell lines.

Laboratory strains of HIV-1, HIV-2, simian immunodeficiency virus, strains with know resistance to indicated drugs, or a low-passage (clinically derived) isolates are used to infect established cell lines by using a specific multiplicity of infection (MOI) of the virus for 1 hour at 37° C. prior to washing the cells and resuspension in medium containing increasing concentrations of drug (or test compound). At 4 to 6 days post-infection, drug-treated and control wells are analyzed for an HIV-1 induced cytopathic effect and/or for the presence of viral reverse transcriptase (RT) or viral p24 antigen in the culture medium (Buckheit and Swanstrom 1991; Ojwang et al. 1994, 1995a, Rando et al. 1995).

Buckheit, R. W., Jr, and Swanstrom, R. I. (1991) AIDS Res. Human Retroviruses 7:295-302.

Ojwang, J. Q., Elbaggari, A., Marshall, H. B., Jayaraman, K., McGrath, M. S., and Rando, R. F. (1994) J. Acquired Immune Deficiency Syndrome 7:560-570.

Rando, R. F., Ojwang, J. O., Elbaggari, A., Reyes, G. R., Tinder, R., McGrath, M. S., and Hogan, M. E., (1995) J. Biol. Chem. 270:1754-1760.

Ojwang, J. O., Buckheit, R. W., Pommier, Y., Mazumder, A., De Vreese, K., Este, J. A., Reymaen, D., Pallansch, L. A., Lackman-Smith, C., Wallace, T. L., De Clercq, E., McGrath, M. S., and Rando, R. F. (1995a) Antimicrobial Agents and Chemotherapy. 39:2426-2435.

HIV-1 infection of PBMCs, CBMCs, and PBL.

Peripheral blood mononuclear cells (PBMCs), Core blood mononuclear cells (CBMCs) and peripheral blood lymphocytes (PBLs) are isolated from the blood of HIV-1 negative and hepatitis B virus-negative (healthy) donors by using the Ficoll-Hypaque density gradient centrifugation technique and are then cultured as described by Gartner and Popovic (1990).

PBMCs or CBMCs (2×10⁵ cells/well) were infected with various isolates of HIV-1 at an MOI of 0.01. After 2 hours at 37C the cells are washed and treated with various concentrations of drug or test compound (Buckheit and Swanstrom 1991; Ojwang et al. 1995a). Seven days post-infection, HIV-1 replication was analyzed by the measuring the levels of RT or p24 in the culture medium.

PBLs (2×10⁵ cells/well) were infected with various isolates of HIV-1 at an MOI of 0.2. This level of infection yields a satisfactory RT activity for the virus control at day 7 post-infection (Buckheit and Swanstrom 1991; Ojwang et al. 1995a). After 2 hours at 37C the cells are washed and treated with various concentrations of drug or test compound (Buckheit and Swanstrom 1991; Ojwang et al. 1995a). Seven days post-infection, HIV-1 replication is analyzed by measuring the levels of viral RT or p24 in the culture medium.

Gartner, S., and Popovic, M. (1990) Virus Isolation and Production, p. 59-63. In A. Aldovini and B. Walker (ed) Techniques in HIV research. Stockton Press, New York.

HIV-1 infection of primary human macrophages.

Human macrophage cultures are established as described by Crowe et al. (1987). Macrophages are cultivated in RPMI 1640 supplemented with 10% human serum. After incubation overnight at 37C the macrophages are infected with HIV-1 at an MOI of 0.1 for 24 hr at 37C in the presence of the indicated amount of test compound (McGrath et al. 1989). On day 7 post-infection the adherent macrophages are washed extensively with phosphate-buffered saline (PBS) and are lysed with detergent. Cytoplasmic HIV-1 p24 levels are then quantitated, and percent inhibition is calculated and compared with that for control infected but untreated cells.

Crowe, S. M., Mills, J., and McGrath, M. S. (1987) AIDS Res and Hum. Retroviruses 3:135-145.

McGrath, M. S. et al. (1989) Proc. Natl. Acad. Sci. USA 86:2844-2848.

HIV-1 Integrase inhibition assays

HIV-1 integrase enzyme assays were performed as described by Ojwang et al. (1995a) and Mazumder et al. (1996). The integrase enzyme is pre-incubated at a final concentration of 200 nM (for HIV-1 or HIV-1 enzyme) or 600 nM (for FIV or SIV derived enzyme) with inhibitor in reaction buffer [50 mM NaCl, 1 mM HEPES, pH 7.5, 50 μM EDTA, 50 μM dithiothereitol, 10% glycerol (w/v), 7.5 mM MnCl₂ (when specified MgCl is used), 0.1 mg/ml bovine serum albumin, 10 mM 2-mercaptoehtanol, 10% dimethyl sulfoxide and 25 mM MOPS, pH 7.2] at 30° C. for 30 min. When magnesium was used as the divalent metal ion, poly(ethylene glycol) was added at a final concentration of 5% to increase the activity as previously described (Engelman and Craigie, 1995). Pre-incubation for 30 min of the enzyme with inhibitor was performed to optimize the inhibitory activity in the 3-processing reaction (Fesen et al. 1994). Then, 20 nM of the 5′-end ³²P-labeled linear oligonucleotide substrate was added, and the incubation was continued for an additional 1 hour. The final reaction volume was 16 μl.

Disintegration reactions (Chow et al. 1992) were performed as above with a Y oligonucleotide (i.e. branched substrate in which the U5 end was integrated into target DNA was used).

Antiviral Activity & Cytotoxicity

| Compound | Structure | IC50 (μM) | CC50 (μM) |
|---|---|---|---|
| 44 | 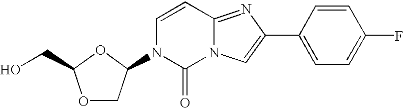 | 0.003 | >100 |
| 46 | 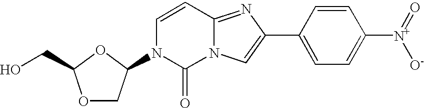 | 0.1 | >100 |
| 48 | 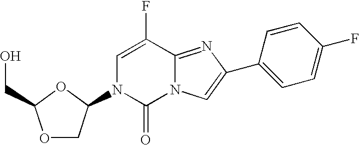 | 0.026 | >100 |
| 49 | 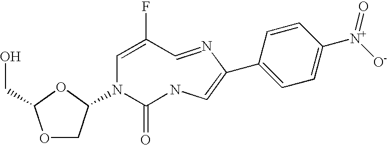 | 0.005 | — |
| 50 | 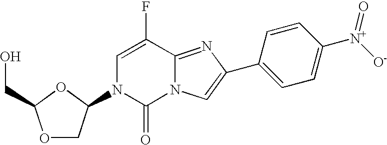 | 0.44 | >100 |
| 58 | 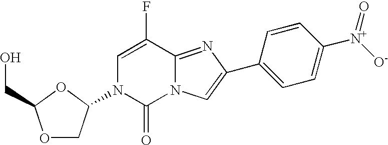 | 60 | >100 |

TABLE 2
Antiviral Activity & Cytotoxicity Using T Cells

| Compound | | | | |
|---|---|---|---|---|
| No. | Structure | IC₅₀ HIV | CC₅₀ | SI (HIV) |
| 30 | 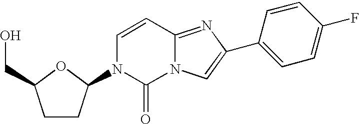 | 5.0 | >100 | >20 |
| 31 | 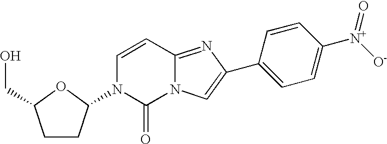 | 9.7 | 45 | 4.6 |
| 32 | 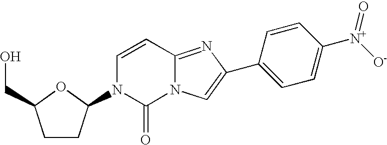 | 7.0 | >100 | >14 |
| 33 | 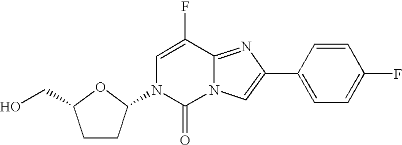 | 4.0 | 90 | 22.5 |
| 36 | 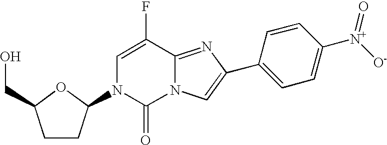 | 1.0 | >100 | >100 |
| 39 | 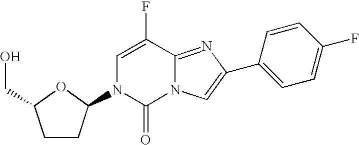 | 48 | >100 | >2.1 |
| 43 | 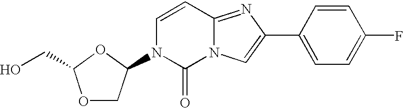 | 0.31 | >100 | >322 |
| 44 | 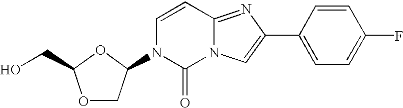 | 0.0032 | >100 | >31250 |
| 45 | 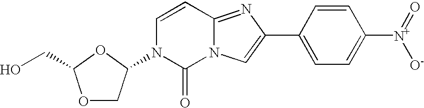 | 0.25 | 10 | 40 |
| 46 | 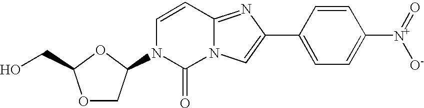 | 0.1 | >100 | >1000 |
| 48 | 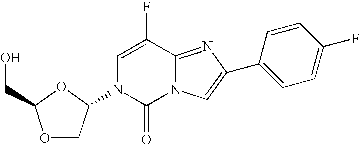 | 0.026 | >100 | >3846 |
| 50 | 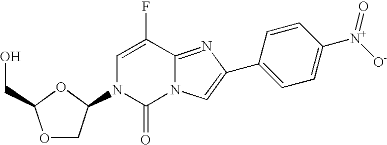 | 0.44 | >100 | >227 |
| 53 | 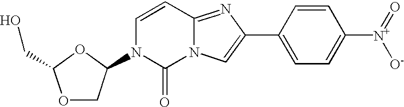 | 0.4 | >10 | >25 |
| 55 | 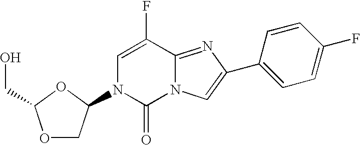 | 48 | >100 | >2.1 |
| 58 | 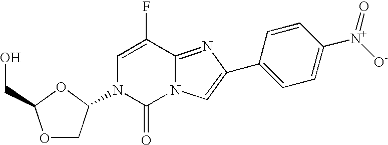 | 60 | >100 | >1.6 |

| | |
|---|---|
| HIV | CC50 |
| IC50 (uM) | (uM) |

| Compound # | MT-2 | PBMCs | MT-2 | PBMCs |
|---|---|---|---|---|
| 50 | 2.8 | | >10 | |
| 49 | 0.13 | 0.54 | 6.5 | <10 |
| | 0.015 (MT-4) | 0.039 | | |
| | 0.24 Jurkat | | | |
| 45 | 3 | | 2 | |
| 60 | | 0.6 | | >10 |
| 62 | | 8 | | >10 |

1. A method for the treatment of viral infections in mammals comprising the administration of a therapeutically effective amount of a compound of formula (I), 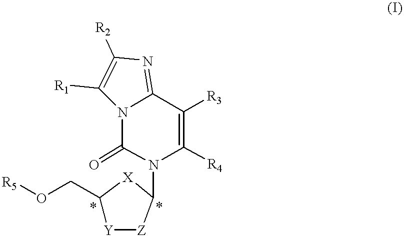
salts or esters of said compound, pharmaceutical acceptable derivatives or pharmaceutically acceptable salts or esters thereof, wherein:
X is oxygen, sulfur or CH₂;
Y and Z are independently selected from sulfur, oxygen, CF₂, C═CH₂ or CH(Rₐ) wherein Rₐ is hydrogen, OH, CN, halogen, N₃, NH₂, SH, C₁₋₆ alkyl, C₁₋₆ alkoxy, C₂₋₆ alkenyl, C₂₋₆ alkynyl, C(O)R_{b}, NHR_{b}, SR_{b} wherein R_{b} is hydrogen, OH, CN, halogen, N₃, NH₂, SH, C₁₋₆ alkyl or C₁₋₆ acyl, C(O)OR_{c} wherein R_{c} is C₁₋₆ alkyl or C₁₋₆ acyl, or Y is CH provided that Z is CH and Y and Z are linked by a double bond;
R₁ is hydrogen, C₁₋₆ alkyl, C₆₋₁₀ aryl or halogen;
R₂ is hydrogen, C₁₋₆ alkyl, C₁₋₆ alcyl, C₆₋₁₀ aryl, C₅₋₁₀ heterocycle, C₂₋₆ acyloxycarbonyl or C₇₋₁₃ aryloxycarbonyl or halogen;
R₃ is hydrogen C₁₋₆ alkyl, C₆₋₁₀ aryl or halogen;
R₄ is hydrogen or halogen;
R₅ is hydrogen; C₁₋₁₀ silylalkyl; C₂₋₁₀ acyl or P(O)(OR₆)O(R′₆) wherein R₆ and R′₆ are independently selected from group H, C₁₋₆ alkyl, C₂₋₆ alkenyl, C₆₋₁₀ aryl, C₇₋₁₁ arylmethyl, C₂₋₇ acyloxymethyl, C₃₋₈ alkoxycarbonyloxymethyl, C₇₋₁₁ aryloyloxymethyl, C₃₋₈S-acyl-2-thioethyl; phosphonophosphate or phosphonodiphosphate.

2. A method according to claim 1 wherein the viral infection is an HIV infection.

3. A method according to claim 2 wherein R₁ and R₄ is hydrogen; X is oxygen or sulfur; Z is CH₂; and the mammal a human.

4. A method according to claim 3 wherein X is oxygen.

5. A method according to claim 3 wherein R₂ is hydrogen, C₆₋₁₀ aryl, C₅₋₁₀ heterocycle, C₁₋₆ alkyl, C₂₋₆ acyloxycarbonyl or halogen.

6. A method according to claim 3 wherein R₃ is selected from hydrogen, fluoro, bromo, chloro or iodo.

7. A method according to claim 6 wherein R₃ is hydrogen or fluoro.

8. A method according to claim 3 wherein R₅ is hydrogen; C₁₋₁₀ silylalkyl; C₂₋₁₀ acyloxy; phosphate; phosphonophosphate or phosphonodiphosphate.

9. A method according to claim 3 wherein Y is oxygen.

10. A method according to claim 3 wherein Y is CH₂.

11. A method according to claim 1 wherein X is oxygen; Y is oxygen or CH₂; R₁ and R₄ are hydrogen; R₃ is hydrogen orfluoro; R₂ is hydrogen, C₆₋₁₀ aryl, C₅₋₁₀ heterocycle, C₁₋₆ alkyl, C₂₋₆ acyloxycarbonyl or halogen; and R₅ is hydrogen, C₁₋₁₀ silylalkyl or C₂₋₁₀ acyloxy.

12. A method according to claim 1 wherein the compound of formula (I) is selected from the group consisting of:
6-[(2S,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;
6-[(2R,5S)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;
6-[(2S,5R)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;
2-(4-NITROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-FLUOROPHENYL)-6-[(5R,2S)-5-HYDROXYMETHYL OXOLAN-2-YL]-5,6-DIHYDRO[1,3]DIAZOLO[1,2-C][1,3]DIAZIN-5-ONE;
8-FLUORO-2-(4-FLUOROPHENYL)-6-[(2R,5S)-5-HYDROXYMETHYL OXOLAN-2-YL]-5,6-DIHYDRO[1,3]-DIAZOLO[1,2-C][1,3]DIAZIN-5-ONE;
8-FLUORO-2-(4-NITROPHENYL)-6-[(5R,2S)-5-HYDROXYMETHYL OXOLAN-2-YL]-5,6-DIHYDRO[1,3]DIAZOLO[1,2-C][1,3]DIAZIN-5-ONE;
8-FLUORO-2-(4-NITROPHENYL)-6-[(2R,5S)-5-HYDROXYMETHYL OXOLAN-2-YL]-5,6-DIHYDRO[1,3]-DIAZOLO[1,2-C][1,3]DIAZIN-5-ONE;
2-(4-FLUOROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
2-(4-NITROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
6-[(2R,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;
8-FLUORO-2-(4-FLUOROPHENYL)-6-[(2S,5S)-5-HYDROXYMETHYL OXOLAN-2-YL]-5,6-DIHYDRO[1,3]-DIAZOLO[1,2-C][1,3]DIAZIN-5-ONE;
8-FLUORO-2-(4-NITROPHENYL)-[(2R,5R)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-6-[(2S,5S)-5-HYDROXYMETHYL OXOLAN-2-YL]-5,6-DIHYDRO[1,3]-DIAZOLO[1,2-C][1,3]DIAZIN-5-ONE;
2-(4-FLUOROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-FLUOROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
L2-(4-NITROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-NITROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-FLUOROPHENYL)-6-[(2S,4S)-5-HYDROXYMETHYL OXOLAN-4-YL]-5,6-DIHYDRO[1,3]-DIAZOLO[1,2-C][1,3]DIAZIN-5-ONE;
8-FLUORO-2-(4-FLUOROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-NITROPHENYL)-6-[(2S,4S)-5-HYDROXYMETHYL OXOLAN-4-YL]-5,6-DIHYDRO[1,3]-DIAZOLO[1,2-C][1,3]DIAZIN-5-ONE;
8-FLUORO-2-(4-NITROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
2-(4-FLUOROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
2-(4-FLUOROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-FLUOROPHENYL)-6-[(2S,4R)-2-HYDROXYMETHYL-1,3-DIOXOLAN-4-YL]-5,6-DIHYDRO[1,3]DIOXOLO[1,2-C][1,3]DIAZIN-5-ONE;
8-FLUORO-2-(4-FLUOROPHENYL)-[(2R,4S)-5-HYDROXYMETHYL OXOLAN-4-YL]-5,6-DIHYDRO[1,3]-DIAZOLO[1,2-C][13]DIAZIN-5-ONE;
8-FLUORO-2-(4-NITROPHENYL)-6-[(2S,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-NITROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE;
4-(6-(2S,5R)-5-HYDROXYMETHYLOXOLAN-2-YL-(5-OXO-5,6-DIHYDRO[1,3-DIAZOLO[1,2-C][1,3]-DIAZIN-2-YL)PHENYLAMMONIUM CHLORIDE;
2-AMINO-3-METHYL-BUTYRIC ACID 4-[2-(4-FLUORO-PHENYL)-5-OXO-IMIDAZO[1,2-C]PYRIMIDIN-6-YL]-[1,3]DIOXOLAN-2-YLMETHYL ESTER; HYDROCHLORIDE SALT;
6-(2-HYDROXYMETHYL-[1,3]DIOXOLAN-4-YL)-2-PYRIDIN-2-YL-6H-IMIDAZO[1,2-C]PYRIMIDIN-5-ONE; HYDROCHLORIDE SALT;
6-(2-HYDROXYMETHYL-[1,3]DIOXOLAN-4-YL)-2-PHENYL-6H-IMIDAZO[1,2-C]PYRIMIDIN-5-ONE;
6-(2-HYDROXYMETHYL-[1,3]DIOXOLAN-4-YL)-2-PYRIDIN-2-YL-6H-IMIDAZO[1,2-C]PYRIMIDIN-5-ONE;
6-(2-HYDROXYMETHYL-[1,3]DIOXOLAN-4-YL)-6H-IMIDAZO[1,2-C]PYRIMIDIN-5-ONE; and
6-(2-HYDROXYMETHYL-(1,3,)DIOXOLAN-4-YL)-3-PYRIDlN-3-YL-6H-IMIDAZO(1,2)PRIMIDIN-5-ONE.

13. A method according to claim 12 wherein the compound of formula (I) is selected from the group consisting of:
2-(4-FLUOROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-FLUOROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
L2-(4-NITROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-NITROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-FLUOROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-FLUOROPHENYL-)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-NITROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-NITROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-NITROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE; and
2-AMINO-3-METHYL-BUTYRIC ACID 4-[2-(4-FLUORO-PHENYL)-5-OXO-IMIDAZO[1,2-C]PYRIMIDIN-6-YL]-[1,3]DIOXOLAN-2-YLMETHYL ESTER; HYDROCHLORIDE SALT.

14. The method according to any of claims 1, 3, 11 or 12 wherein the compound of formula (I) and additional therapeutic agents are administered sequentially.

15. The method according to any of claims 1, 3, 11 or 12 wherein the compounds and additional therapeutic agents are administered simultaneously.

16. A pharmaceutical formulation effective against viral infections comprising a pharmaceutically effective amount of a compound of formula (I) 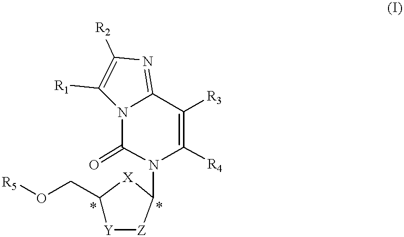
salts or esters of said compound, pharmaceutical acceptable derivatives or pharmaceutically acceptable salts or esters thereof, wherein:
X is oxygen, sulfur or CH₂,
Y and Z are independently selected from sulfur, oxygen, CF₂, C═CH₂ or CH(Rₐ) wherein Rₐ is hydrogen, OH, CN, halogen, N₃, NH₂, SH, C₁₋₆ alkyl, C₁₋₆ alkoxy, C₂₋₆ alkenyl, C₂₋₆ alkynyl, C(O)R_{b}, NHR_{b}, SR_{b} wherein R_{b} is hydrogen, OH, CN, halogen, N₃, NH₂, SH, C₁₋₆ alkyl or C₁₋₆ acyl, C(O)OR_{c} wherein R_{c} is C₁₋₆ alkyl or C₁₋₆ acyl, or Y is CH provided that Z is CH and Y and Z are linked by a double bond,
R₁ is hydrogen, C₁₋₆ alkyl, C₆₋₁₀ aryl or halogen;
R₂ is hydrogen, C₁₋₆ alkyl, C₁₋₆ acyl, C₆₋₁₀ aryl, C₅₋₁₀ heterocycle, C₂₋₆ acyloxycarbonyl or C₇₋₁₃ aryloxycarbonyl or halogen;
R₃ is hydrogen C₁₋₆ alkyl, C₆₋₁₀ aryl or halogen;
R₄ is hydrogen or halogen;
R₅ is hydrogen; C₁₋₁₀ silylalkyl; C₂₋₁₀ acyl or P(O)(OR₆)O(R′₆) wherein R₆ and R′₆ are independently selected from group H, C₁₋₆ alkyl, C₂₋₆ alkenyl, C₆₋₁₀ aryl, C₇₋₁₁ arylmethyl, C₂₋₇ acyloxymethyl, C₃₋₈ alkoxycarbonyloxymethyl, C₇₋₁₁ aryloyloxymethyl, C₃₋₈ S-acyl-2-thioethyl; phosphonophosphate or phosphonodiphosphate.

17. A pharmaceutical formulation according to claim 16 wherein the viral infection is an HIV infection.

18. A pharmaceutical formulation according to claim 16 additionally comprising a further therapeutic agent.

19. A pharmaceutical formulation according to claim 18 wherein the further therapeutic agent is active against an HIV infection.

20. A pharmaceutical formulation according to claim 19 wherein the therapeutic agent is selected form the group consisting of epivir, DAPD, FTC, AZT, nevirapine, DMP-226, nelfinavir, indinavir, delavirdine, MKC-442, abacavir, saquinavir, ritonavir, acyclovir, d4T, ddC, and ddl.

21. A pharmaceutical formulation according to claim 16 wherein X is oxygen; Y is oxygen or CH₂; R₁ and R₄ are hydrogen; R₃ is hydrogen or fluoro; R₂ is hydrogen, C₆₋₁₀ aryl, C₅₋₁₀ heterocycle, C₁₋₆ alkyl, C₂₋₆ acyloxycarbonyl or halogen; and R₅ is hydrogen, C₁₋₁₀ silylalkyl or C₂₋₁₀ acyloxy.

22. A pharmaceutical formulation according to claims 16 or 18 wherein the compound of formula (I) is selected from the group consisting of:
6-[(2S,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;
6-[(2R,5S)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;
6-[(2S,5R)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;
2-(4-NITROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-FLUOROPHENYL)-6-[(5R,2S)-5-HYDROXYMETHYLOXOLAN-2-YL]-5,6-DIHYDRO[1,3]DIAZOLO[1,2-C][1,3]DIAZIN-5-ONE;
8-FLUORO-2-(4-FLUOROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-6-[(5R,2S)-5-HYDROXYMETHYLOXOLAN-2-YL]-5,6-DIHYDRO[1,3]DIAZOLO[1,2-C][1,3]DIAZIN-5-ONE;
8-FLUORO-2-(4-NITROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
2-(4-FLUOROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
2-(4-NITROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
6-[(2R,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;
8-FLUORO-2-(4-FLUOROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-[(2R,5R)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
2-(4-FLUOROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-FLUOROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
L2-(4-NITROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-NITROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-FLUOROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-FLUOROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-NITROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-NITROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
2-(4-FLUOROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
2-(4-FLUOROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-FLUOROPHENYL)-6-[(2S,4R)-2-HYDROXYMETHYL-1,3-DIOXOLAN-4-YL]-5,6-DIHYDRO[1,3]DIOXOLO[1,2-C][1,3]DIAZIN-5-ONE;
8-FLUORO-2-(4-FLUOROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-NITROPHENYL)-6-[(2S,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-NITROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE;
4-(6-(2S,5R)-5-HYDROXYMETHYLOXOLAN-2-YL-(5-OXO-5,6-DIHYDRO[1,3-DIAZOLO[1,2-C][1,3]-DIAZIN-2-YL)PHENYLAMMONIUM CHLORIDE;
2-AMINO-3-METHYL-BUTYRIC ACID 4-[2-(4-FLUORO-PHENYL)-5-OXO-IMIDAZO[1,2-C]PYRIMIDIN-6-YL]-[1,3]DIOXOLAN-2-YLMETHYL ESTER; HYDROCHLORIDE SALT;
6-(2-HYDROXYMETHYL-[1,3]DIOXOLAN-4-YL)-2-PYRIDIN-2-YL-6H-IMIDAZO[1,2-C]PYRIMIDIN-5-ONE; HYDROCHLORIDE SALT;
6-(2-HYDROXYMETHYL-[1,3]DIOXOLAN-4-YL)-2-PHENYL-6H-IMIDAZO[1,2-C]PYRIMIDIN-5-ONE;
6-(2-HYDROXYMETHYL-[1,3]DIOXOLAN-4-YL)-2-PYRIDIN-2-YL-6H-IMIDAZO[1,2-C]PYRIMIDIN-5-ONE;
6-(2-HYDROXYMETHYL-[1,3]DIOXOLAN-4-YL)-6H-IMIDAZO[1,2-C]PYRIMIDIN-5-ONE; and
6-(2-HYDROXYMETHYL-(1,3,)DIOXOLAN-4-YL)-3-PYRIDIN-3-YL-6H-IMIDAZO(1,2)PRIMIDIN-5-ONE.

23. The use of a compound of formula (I) 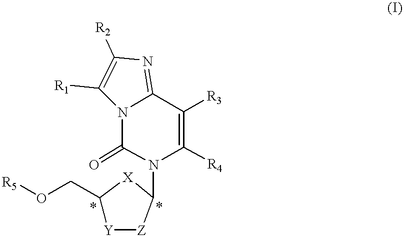
salts or esters of said compound, pharmaceutical acceptable derivatives or pharmaceutically acceptable salts or esters thereof, wherein:
X is oxygen, sulfur or CH₂,
Y and Z are independently selected from sulfur, oxygen, CF₂,
C═CH₂ or CH(Rₐ) wherein Rₐ is hydrogen, OH, CN, halogen, N₃, NH₂, SH, C₁₋₆ alkyl, C₁₋₆ alkoxy, C₂₋₆ alkenyl, C₂₋₆ alkynyl, C(O)R_{b}, NHR_{b}, SR_{b} wherein R_{b} is hydrogen, OH, CN, halogen, N₃, NH₂, SH, C₁₋₆ alkyl or C₁₋₆ acyl, C(O)OR_{c} wherein R_{c} is C₁₋₆ alkyl or C₁₋₆ acyl, or Y is CH provided that Z is CH and Y and Z are linked by a double bond,
R₁ is hydrogen, C₁₋₆ alkyl, C₆₋₁₀ aryl or halogen;
R₂ is hydrogen, C₁₋₆ alkyl, C₁₋₆ alcyl, C₆₋₁₀ aryl, C₅₋₁₀ heterocycle, C₂₋₆ acyloxycarbonyl or C₇₋₁₃ aryloxycarbonyl or halogen;
R₃ is hydrogen C₁₋₆ alkyl, C₆₋₁₀ aryl or halogen;
R₄ is hydrogen or halogen;
R₅ is hydrogen; C₁₋₁₀ silylalkyl; C₂₋₁₀ acyl or P(O)(OR₆)O(R′₆) wherein R₆ and R′₆ are independently selected from group H, C₁₋₆ alkyl, C₂₋₆ alkenyl, C₆₋₁₀ aryl, C₇₋₁₁ arylmethyl, C₂₋₇ acyloxymethyl, C₃₋₈ alkoxycarbonyloxymethyl, C₇₋₁₁ aryloyloxymethyl, C₃₋₈ S-acyl-2-thioethyl; phosphonophosphate or phosphonodiphosphate;
in the manufacture of a medicament for the treatment of human immunodeficiency virus.

24. A commercial package for the treatment of an HIV infection in mammals, said package comprising a pharmaceutical agent therapeutically effective against the HIV infection, said pharmaceutical agent comprised in said commercial package a compound of formula (I): 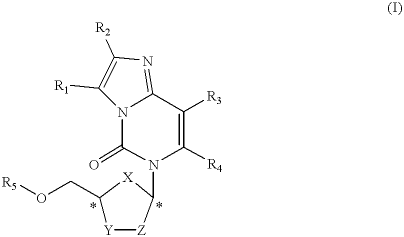
salts or esters of said compound, pharmaceutical acceptable derivatives or pharmaceutically acceptable salts or esters thereof, wherein:
X is oxygen, sulfur or CH₂,
Y and Z are independently selected from sulfur, oxygen, CF₂, C═CH₂ or CH(Rₐ) wherein Rₐ is hydrogen, OH, CN, halogen, N₃, NH₂, SH, C₁₋₆ alkyl, C₁₋₆ alkoxy, C₂₋₆ alkenyl, C₂₋₆ alkynyl, C(O)R_{b}, NHR_{b}, SR_{b} wherein R_{b} is hydrogen, OH, CN, halogen, N₃, NH₂, SH, C₁₋₆ alkyl or C₁₋₆ acyl, C(O)OR_{c} wherein R_{c} is C₁₋₆ alkyl or C₁₋₆ acyl, or Y is CH provided that Z is CH and Y and Z are linked by a double bond,
R₁ is hydrogen, C₁₋₆ alkyl, C₆₋₁₀ aryl or halogen;
R₂ is hydrogen, C₁₋₆ alkyl, C₁₋₆ acyl, C₆₋₁₀ aryl, C₅₋₁₀ heterocycle, C₂₋₆ acyloxycarbonyl or C₇₋₁₃ aryloxycarbonyl or halogen;
R₃ is hydrogen C₁₋₆ alkyl, C₆₋₁₀ aryl or halogen;
R₄ is hydrogen or halogen;
R₅ is hydrogen; C₁₋₁₀ silylalkyl; C₂₋₁₀ acyl or P(O)(OR₆)O(R₆) wherein R₆ and R₆ are independently selected from group H, C₁₋₆ alkyl, C₂₋₆ alkenyl, C₆₋₁₀ aryl, C₇₋₁₁ arylmethyl, C₂₋₇ acyloxymethyl, C₃₋₈ alkoxycarbonyloxymethyl, C₇₋₁₁ aryloyloxymethyl, C₃₋₈ S-acyl-2-thioethyl; phosphonophosphate or phosphonodiphosphate.

25. A commercial package according to claim 22 wherein the compound of formula (I) is selected from the group consisting of:
6-[(2S,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;
6-[(2R,5S)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;
6-[(2S,5R)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;
2-(4-NITROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-FLUOROPHENYL)-6-[(5R,2S)-5-HYDROXYMETHYLOXOLAN-2-YL]-5,6-DIHYDRO[1,3]DIAZOLO[1,2-C][1,3]DIAZIN-5-ONE;
8-FLUORO-2-(4-FLUOROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-6-[(5R,2S)-5-HYDROXYMETHYLOXOLAN-2-YL]-5,6-DIHYDRO[1,3]DIAZOLO[1,2-C][1,3]DIAZIN-5-ONE;
8-FLUORO-2-(4-NITROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
2-(4-FLUOROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
2-(4-NITROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
6-[(2R,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;
8-FLUORO-2-(4-FLUOROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-[(2R,5R)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-6-[(2S,5S)-5-HYDROXYMETHYLOXOLAN-2-YL]-5,6-DIHYDRO[1,3]-DIAZOLO[1,2-C][1,3]DiAZIN-5-ONE;
2-(4-FLUOROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-FLUOROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
L2-(4-NITROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-NITROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-FLUOROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-FLUOROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-NITROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-NITROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
2-(4-FLUOROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
2-(4-FLUOROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-FLUOROPHENYL)-6-[(2S,4R)-2-HYDROXYMETHYL-1,3-DIOXOLAN-4-YL]-5,6-DIHYDRO[1,3]DIOXOLO[1,2-C][1,3]DIAZIN-5-ONE;
8-FLUORO-2-(4-FLUOROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-NITROPHENYL)-6-[(2S,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-NITROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE;
4-(6-(2S,5R)-5-HYDROXYMETHYLOXOLAN-2-YL-(5-OXO-5,6-DIHYDRO[1,3-DIAZOLO[1,2-C][1,3]-DIAZIN-2-YL)PHENYLAMMONIUM CHLORIDE;
2-AMINO-3-METHYL-BUTYRIC ACID 4-[2-(4-FLUORO-PHENYL)-5-OXO-IMIDAZO[1,2-C]PYRIMIDIN-6-YL]-[1,3]DIOXOLAN-2-YLMETHYL ESTER; HYDROCHLORIDE SALT;
6-(2-HYDROXYMETHYL-[1,3]DIOXOLAN-4-YL)-2-PYRIDIN-2-YL-6H-IMIDAZO[1,2-C]PYRIMIDIN-5-ONE; HYDROCHLORIDE SALT;
6-(2-HYDROXYMETHYL-[1,3]DIOXOLAN-4-YL)-2-PHENYL-6H-IMIDAZO[1,2-C]PYRIMIDIN-5-ONE;
6-(2-HYDROXYMETHYL-[1,3]DIOXOLAN-4-YL)-2-PYRIDIN-2-YL-6H-IMIDAZO[1,2-C]PYRIMIDIN-5-ONE;
6-(2-HYDROXYMETHYL-[1,3]DIOXOLAN-4-YL)-6H-IMIDAZO[1,2-C]PYRIMIDIN-5-ONE; and
6-(2-HYDROXYMETHYL-(1,3,)DIOXOLAN-4-YL)-3-PYRIDIN-3-YL-6H-IMIDAZO(1,2)PRIMIDIN-5-ONE.

26. An imidazopyrimidine nucleoside of formula (I) 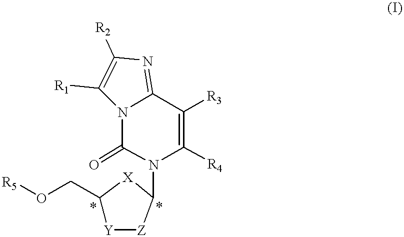
salts or esters of said compound, pharmaceutical acceptable derivatives or pharmaceutically acceptable salts or esters thereof, wherein:
X is oxygen;
Y is oxygen or CH₂;
R₁ and R₄ are hydrogen;
R₃ is hydrogen or fluoro;
R₂ is hydrogen, C₆₋₁₀ aryl, C₅₋₁₀ heterocycle, C₁₋₆ alkyl, C₂₋₆ acyloxycarbonyl or halogen; and
R₅ is hydrogen, C₁₋₁₀ silylalkyl or C₂₋₁₀ acyl.

27. A compound according to claim 26 wherein R₂ is hydrogen, C₆₋₁₀ aryl or C₅₋₁₀ heterocycle.

28. A compound according to claim 27 wherein R₂ is hydrogen; phenyl optionally substituted with nitro, hydroxy, halogen, amino, C₁₋₆ alkyl, C₂₋₁₀ acyl, C₂₋₁₀ alkyloxy; napthyl optionally substituted with nitro, hydroxy, halogen, amino, C₁₋₆ alkyl, C₂₋₁₀ acyl, C₂₋₁₀ alkyloxy; pyridyl; pyrimidinyl; thienyl; pyrazinyl; imidazoyl; pyrrolyl; indazolyl; or purinyl.

29. A compound according to claim 26 wherein R₃ is halogen or hydrogen.

30. A compound according to claim 29 wherein R₃ is fluoro, bromo, iodo, chloro or hydrogen.

31. A compound according to claim 26 wherein R₅ is hydrogen or C₂₋₁₀ acyl.

32. A compound selected from the group consisting of
6-[(2S,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;
6-[(2R,5S)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;
6-[(2S,5R)-5-HYDROXYMETHYLOXOLAN-2-YL]-2-(4-NITROPHENYL)-5,6-DIHYDRO[1,3]-DIAZOLO[1,2-C][1,3]DIAZIN-5-ONE;
2-(4-NITROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-FLUOROPHENYL)-6-[(5R,2S)-5-HYDROXYMETHYLOXOLAN-2-YL]-5,6-DIHYDRO[1,3]DIAZOLO[1,2-C][1,3]DIAZIN-5-ONE;
8-FLUORO-2-(4-FLUOROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-6-[(5R,2S)-5-HYDROXYMETHYLOXOLAN-2-YL]-5,6-DIHYDRO[1,3]DIAZOLO[1,2-C][1,3]DIAZIN-5-ONE;
8-FLUORO-2-(4-NITROPHENYL)-6-[(2R,5S)-5-HYDROXYMETHYLOXOLAN-2-YL]-5,6-DIHYDRO[1,3]-DIAZOLO[1,2-C][1,3]DIAZIN-5-ONE;
2-(4-FLUOROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
2-(4-NITROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
6-[(2R,5R)-5-HYDROXYMETHYLOXOLAN-2-YL]-2-(4-FLUOROPHENYL)-5,6-DIHYDRO[1,3]-DIAZOLO[1,2-C][1,3]DIAZIN-5-ONE;
8-FLUORO-2-(4-FLUOROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-[(2R,5R)-5-HYDROXYMETHYLOXOLAN-2-YL]-5,6-DIHYDRO[1,3]-DIAZOLO[1,2-C][1,3]DIAZIN-5-ONE;
8-FLUORO-2-(4-NITROPHENYL)-6-[(2S,5S)-5-HYDROXYMETHYLOXOLAN-2-YL]-5,6-DIHYDRO[1,3]-DIAZOLO[1,2-C][1,3]DIAZIN-5-ONE;
2-(4-FLUOROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-FLUOROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
L2-(4-NITROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-NITROPHENYL)-6-[(2R,4R)-2-HYDROXYMETHYL-1,3-DIOXOLAN4-YL]-5,6-DIHYDRO[1,3]DIAZOLO[1,2-C][1,3]DIAZIN-5-ONE;
8-FLUORO-2-(4-FLUOROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-FLUOROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-NITROPHENYL)-6-[(2S,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-NITROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE;
2-(4-FLUOROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
2-(4-FLUOROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2S,4R)-5-HYDROXYMETHYL-1,3-DIOXOLAN-4-YL]-5,6-DIHYDRO[1,3]-DIAZOLO[1,2-C][1,3]DIAZIN-5-ONE;
2-(4-NITROPHENYL)-6-[(2R,4S)-5-HYDROXYMETHYL-1,3-DIOXOLAN-4-YL]-5,6-DIHYDRO[1,3]-DIAZOLO[1,2-C][1,3]DIAZIN-5-ONE;
8-FLUORO-2-(4-FLUOROPHENYL)-6-[(2S,4R)-2-HYDROXYMETHYL-1,3-DIOXOLAN-4-YL]-5,6-DIHYDRO[1,3]DIOXOLO[1,2-C][1,3]DIAZIN-5-ONE;
8-FLUORO-2-(4-FLUOROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE;
8-FLUORO-2-(4-NITROPHENYL)-6-[(2S,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-NITROPHENYL)-[(2R,4S)-5-HYDROXYMETHYLOXOLAN-4-YL]-5,6-DIHYDRO[1,3]-DIAZOLO[1,2-C][1,3]DIAZIN-5-ONE;
4-(6-(2S,5R)-5-HYDROXYMETHYLOXOLAN-2-YL-(5-OXO-5,6-DIHYDRO[1,3-DIAZOLO[1,2-C][1,3]-DIAZIN-2-YL)PHENYLAMMONIUM CHLORIDE;
2-AMINO-3-METHYL-BUTYRIC ACID 4-[2-(4-FLUORO-PHENYL)-5-OXO-IMIDAZO[1,2-C]PYRIMIDIN-6-YL]-[1,3]DIOXOLAN-2-YLMETHYL ESTER; HYDROCHLORIDE SALT;
6-(2-HYDROXYMETHYL-[1,3]DIOXOLAN-4-YL)-2-PYRIDIN-2-YL-6H-IMIDAZO[1,2-C]PYRIMIDIN-5-ONE; HYDROCHLORIDE SALT;
6-(2-HYDROXYMETHYL-[1,3]DIOXOLAN-4-YL)-2-PHENYL-6H-IMIDAZO[1,2-C]PYRIMIDIN-5-ONE;
6-(2-HYDROXYMETHYL-[1,3]DIOXOLAN-4-YL)-2-PYRIDIN-2-YL-6H-IMIDAZO[1,2-C]PYRIMIDIN-5-ONE;
6-(2-HYDROXYMETHYL-[1,3]DIOXOLAN-4-YL)-6H-IMIDAZO[1,2-C]PYRIMIDIN-5-ONE; and
6-(2-HYDROXYMETHYL-(1,3,)DIOXOLAN-4-YL)-3-PYRIDIN-3-YL-6H-IMIDAZO(1,2)PRIMIDIN-5-ONE.

33. A compound selected from the group consisting of:
2-(4-FLUOROPHENYL)-6-[(2S,4S)-2-HYDROXYMETHYL-1,3-DIOXOLAN-4-YL]-5,6-DIHYDRO[1,3]DIAZOLO[1,2-C][1,3]DIAZIN-5-ONE;
2-(4-FLUOROPHENYL)-6-[(2R,4R)-2-HYDROXYMETHYL-1,3-DIOXOLAN-4-YL]-5,6-DIHYDRO[1,3]DIAZOLO[1,2-C][1,3]DIAZIN-5-ONE;
8-FLUORO-2-(4-FLUOROPHENYL)-[(2R,4R)-2-HYDROXYMETHYL-1,3-DIOXOLAN-4-YL]-5,6-DIHYDRO[1,3]-DIAZOLO[1,2-C][1,3]DIAZIN-5-ONE; and
8-FLUORO-2-(4-NITROPHENYL)-6-[(2S,4S)-5-HYDROXYMETHYL OXOLAN-4-YL]-5,6-DIHYDRO[1,3]-DIAZOLO[1,2-C].

34. A pharmaceutical formulation effective against viral infections comprising a pharmaceutically effective amount of compound of formula (I) according to claim 26 and a pharmaceutically acceptable carrier.

35. A method for the treatment or prophylaxis of an HIV infection in a mammal, comprising administering to said mammal an effective amount of a compound of formula (I) according to claim 26.
